(19) United States
(12) Patent Application Publication
(10) Pub. No.: US 2025/0321205 A1
(43) Pub. Date: Oct. 16, 2025

(54) STRUCTURED MICROGEL ELECTROPHORETIC ARRAYS FOR RAPID MULTIPLEX NUCLEIC ACID DETECTION

(71) Applicant: ADOR DIAGNOSTICS LTD., Limassol (CY)

(72) Inventors: Vladimir HURGIN, Gan Yavne (IL); Nives HODKO, Poway, CA (US); Dalibor HODKO, Poway, CA (US); Zuxu YAO, San Diego, CA (US); Michal SIMOVITCH ETKES, Jerusalem (IL); Eran ZAHAVY, Rehovot (IL)

(21) Appl. No.: 19/251,173

(22) Filed: Jun. 26, 2025

Related U.S. Application Data
(63) Continuation-in-part of application No. 16/958,403, filed on Jun. 26, 2020, now abandoned.

(51) Int. Cl. G01N 27/447; C12Q 1/6855; G01N 21/64; G01N 33/53; G01N 33/569

(52) U.S. Cl. CPC G01N 27/44726; C12Q 1/6855; G01N 21/6486; G01N 27/44747; G01N 33/5308; G01N 33/56916; G01N 33/56944; G01N 2333/245; G01N 2333/26; G01N 2333/285; G01N 2333/315

(57) ABSTRACT

Methods and devices are disclosed for rapid, multiplex molecular detection of diverse nucleic acid target molecules. The invention features an electrophoretic array with immobilized hydrogel microgel deposits. Each deposit comprises a three-dimensional, cross-linked polymer matrix containing an immobilized affinity-binding molecule and a porogen-derived pore network. This structure is configured for rapid molecular transport of nucleic acids (e.g., up to 800 bp), providing a localized environment for target capture, ligation of linear Rolling Circle Amplification (RCA) probes, and RCA. Target-specific components are anchored within distinct microgels for multiplexing. Electric fields enhance transport, reaction kinetics, and amplicon concentration. Detection is achieved in under 20 minutes. The specifically structured and fabricated microgels improve detection speed, sensitivity, and applicability to multiple different targets.

### CROSS-REFERENCE TO RELATED APPLICATIONS

This application is a Continuation-in-Part of U.S. patent application Ser. No. 16/958,403, filed Jun. 26, 2020, which is a U.S. National Phase entry under 35 U.S.C. 371 of PCT Patent Application No. PCT/IL2018/051400 filed Dec. 27, 2018, claiming priority based on U.S. Provisional Patent Application No. 62/610,997 filed Dec. 28, 2017, the disclosure of which is hereby incorporated by reference and priority of which is hereby claimed. All of these earlier applications are incorporated by reference in their entirety.

### FIELD OF THE INVENTION

The present invention pertains generally to the field of molecular diagnostics and analysis. More specifically, the invention relates to methods, devices, and systems for the rapid and sensitive detection of nucleic acid target molecules, including multiplex detection of a plurality of different target molecules. Even more particularly, the invention concerns electrophoretic arrays incorporating structured hydrogel microgel deposits that provide optimized localized environments for nucleic acid amplification, such as Rolling Circle Amplification (RCA), and subsequent detection.

### BACKGROUND

The ability to quickly and accurately identify specific nucleic acid sequences is paramount in numerous applications, most notably in clinical diagnostics for the timely identification of pathogens (such as bacteria or viruses), genetic predispositions, and disease biomarkers. Traditional nucleic acid detection methods, such as the Polymerase Chain Reaction (PCR) often followed by complex downstream analysis, while powerful, typically involve time-consuming thermal cycling, require sophisticated laboratory infrastructure, may involve multi-step sample preparation and have technical limitation for high multiplexing of different nucleic acid target molecules. These limitations can hinder their application in settings requiring rapid turnaround times, such as point-of-care diagnostics or emergency situations.

Isothermal amplification techniques, including Rolling Circle Amplification (RCA), have emerged as promising alternatives. RCA employs a circular DNA template and a strand-displacing polymerase to generate a large number of copies of the template sequence at a constant temperature, thereby simplifying instrumentation requirements. The specificity of RCA can be significantly enhanced through the use of “padlock probes,” which are linear oligonucleotide probes that are circularized by a ligase enzyme only when their ends are brought together by correct hybridization to a specific target nucleic acid sequence. The resulting circular molecule then serves as the template for amplification.

To further enhance the utility of such amplification techniques, particularly for detecting multiple targets from a single sample, array-based formats have been explored. Microarrays or biochips featuring spatially distinct reaction sites allow for parallel analysis. Electrophoretic arrays, which employ electric fields to actively transport and concentrate charged biomolecules like DNA and RNA at specific locations, have been investigated to accelerate various steps in the detection process, including target capture, hybridization, and the concentration of amplification products. This can lead to faster reaction kinetics and improved detection limits.

However, the development and implementation of integrated systems for rapid, multiplexed nucleic acid detection on such platforms have faced challenges.
and there remains a continuous need to improve the robustness, speed, and breadth of applicability of such diagnostic systems. Specifically, optimizing the micro-environment where these sensitive biochemical reactions occur can play a critical role in overcoming previous limitations.

The present invention addresses these ongoing needs by providing advanced methods and devices centred on the use of hydrogel microgel deposits with specifically defined three-dimensional porous structures. These structures are engineered to create optimized localized reaction environments within an electrophoretic array. This improved microgel architecture facilitates rapid electrokinetic transport of target molecules and reagents into the microgel, enhances the efficiency of target capture, ligation, and rolling circle amplification within the confined and optimized space, and allows for effective concentration and retention of the generated amplicons.

The parent U.S. application Ser. No. 16/958,403 (the '403 application), the disclosure of which is incorporated herein by reference in its entirety, describes methods of rapidly detecting nucleic acid target molecules using RCA on electrophoretic arrays that include microgel regions. Concerns were raised regarding the scope of enablement for rapidly detecting “at least one nucleic acid target molecule from a plurality of nucleic acid target molecules” across a broad range of targets, particularly when the specification primarily exemplified meningitis pathogens. The predictability of the art and the amount of experimentation that might be required to apply the claimed methods to different nucleic acid targets were also questioned.

By focusing on these specific structural and functional enhancements of the microgel deposits, the present invention aims to provide broader enablement, improved predictability, and enhanced performance for the rapid and multiplexed detection of a diverse range of nucleic acid targets, including those newly exemplified herein, thereby addressing concerns raised during the prosecution of the parent application and advancing the art of rapid molecular diagnostics.

### SUMMARY OF THE INVENTION

The present invention is directed to improved methods and devices for the rapid, sensitive, and multiplexed detection of nucleic acid target molecules. The invention particularly concerns the use of electrophoretic arrays comprising hydrogel microgel deposits with defined structural characteristics that create optimized localized reaction environments, thereby enhancing the efficiency and speed of nucleic acid amplification, such as Rolling Circle Amplification (RCA), and subsequent detection. These advancements aim to address challenges in the art related to achieving broad applicability, predictability, and very rapid detection across a plurality of diverse target types.

According to a first aspect of the present invention, there is provided an electrophoretic array device for rapid, multiplex detection of a plurality of different pre-defined potential nucleic acid target molecules, the device comprising:
(1) a substrate comprising a plurality of electrodes defining discrete electrode locations; and
(2) a plurality of hydrogel microgel deposits immobilized at said discrete electrode locations, each said hydrogel microgel deposit defining a microgel region and comprising a three-dimensional hydrogel matrix, said matrix comprising:
   (a) a cross-linked polymer;
   (b) an immobilized affinity-binding molecule; and
   (c) a porogen-derived pore network defining interconnected void spaces within said matrix;
   wherein said hydrogel matrix is configured to provide a localized reaction environment for enzymatic reactions and rapid molecular transport of nucleic acid molecules up to at least 800 base pairs in length, and for concentration of reaction products; and
   wherein at least a subset of said hydrogel microgel deposits each contain target-specific components pre-anchored within its said hydrogel matrix via said affinity-binding molecule, said target-specific components being different between at least two microgel deposits in said subset, enabling specific capture or initiation of nucleic acid amplification for said plurality of potentially different pre-defined nucleic acid target molecules.

In one embodiment of this aspect, said hydrogel microgel deposits are formed by UV curing a spotted polymerizable hydrogel solution that includes monomers for said cross-linked polymer, a porogen for forming said porogen-derived pore network, said affinity-binding molecule or precursors thereof, and a photoinitiator.

According to an additional embodiment, said monomers comprise acrylamide and N,N′-methylenebisacrylamide (BIS), said cross-linked polymer is polyacrylamide, and said affinity-binding molecule is modified streptavidin.

In some embodiments, said porogen-derived pore network defining inter-connected void spaces comprises pores with observed diameters that can range from approximately 350 nm to approximately 5000 nm, facilitating transport of larger nucleic acid molecules. In a specific embodiment, said porogen-derived pore network is further characterized by an effective average pore diameter in the range of 200 nm to 400 nm, which is found to be particularly effective for balancing the entry of nucleic acid targets and enzymes with the retention of generated amplicons.

In a further embodiment, said hydrogel matrix of at least a portion of said deposits contains pre-anchored target-specific components specific for nucleic acid targets selected from the group consisting of Neisseria meningitidis, Klebsiella pneumoniae, Listeria monocytogenes, Haemophilus influenzae group B, Escherichia coli, Group B Streptococcus, and combinations thereof. In certain embodiments, said hydrogel microgel deposits are in a dehydrated state suitable for room-temperature storage and are configured to rehydrate upon contact with an aqueous solution.

According to a second aspect of the present invention, there is provided a method of rapidly detecting in a solution the presence of at least one nucleic acid target molecule from a plurality of different pre-defined potential nucleic acid target molecules. The method comprises:
(A) Providing an electrophoretic array device according to the first aspect (including any of its described embodiments regarding pore size);
(B) Introducing said solution, potentially containing one or more of said plurality of different pre-defined nucleic acid target molecules, to said electrophoretic array device, causing said solution to contact said hydrogel microgel deposits having said porogen-derived pore network defining interconnected void spaces;
(C) Utilizing electric fields to drive said at least one nucleic acid target molecule present in said solution into the interconnected void spaces of the porous hydrogel matrix of one or more specific hydrogel microgel deposits of said device containing corresponding nucleic acid target-specific components;
(D) Within at least one said specific hydrogel microgel deposit, forming a ligation-competent complex involving a said nucleic acid target molecule, a linear rolling circle amplification (RCA) probe specific to said nucleic acid target molecule, and at least one primer, wherein said complex formation occurs within the localized reaction environment provided by said hydrogel microgel deposit;
(E) Ligating said linear RCA probe within said complex to form a circular RCA probe template within said hydrogel microgel deposit;
(F) Performing rolling circle amplification using said circular RCA probe template within said hydrogel microgel deposit by introducing at least a polymerase enzyme and nucleotides, wherein electric fields are applied to said plurality of microgel regions during said rolling circle amplification to enhance reaction kinetics within the porous hydrogel matrix and concentrate generated amplicons within said hydrogel microgel deposit; and
(G) Detecting the presence of amplified RCA products concentrated within said at least one hydrogel microgel deposit, thereby detecting the presence of said at least one nucleic acid target molecule, wherein said detecting is completed within a time period of between approximately 8 minutes and 20 minutes from said introducing said solution.

In one embodiment of this second aspect, the cross-linked polymer of the hydrogel matrix of the device is polyacrylamide formed from acrylamide and N,N′-methylenebisacrylamide (BIS) monomers. In another embodiment, the immobilized affinity-binding molecule in the hydrogel microgel deposits of the device is modified streptavidin.

According to a further embodiment, the hydrogel microgel deposits of the device are formed by UV curing a polymerizable solution containing a photoinitiator. In yet further embodiments, said porogen-derived pore network defining interconnected void spaces in the hydrogel microgel deposits of the device enhances the accessibility of said target-specific components pre-anchored within said hydrogel matrix to said at least one nucleic acid target molecule. In a specific embodiment building on the use of modified streptavidin, the target-specific components are biotinylated oligonucleotides selected from the group consisting of target-specific capture probes, primers specific to a unique barcode sequence present on a corresponding linear RCA probe, and target-specific linear RCA probes, anchored via said modified streptavidin.

In some embodiments, said target-specific components pre-anchored within said hydrogel matrix in at least one microgel region of the device comprise target-specific capture probes. Alternatively, in other embodiments, said target-specific components pre-anchored within said hydrogel matrix in at least one microgel region of the device comprise primers specific to a unique barcode sequence present on a corresponding linear RCA probe. In a particular embodiment thereof, said primers are pre-hybridized to said linear RCA probes within said hydrogel matrix of the device prior to introducing said solution. In still other embodiments, said target-specific components pre-anchored within said hydrogel matrix in at least one microgel region of the device comprise target-specific linear RCA probes.

According to an additional embodiment, the target-specific components pre-anchored within said hydrogel matrix in at least one microgel region of the device comprise forward primers and reverse primers for RCA. In still additional embodiment, the solution introduced to said array device comprises the linear RCA probe and said at least one primer. Said target-specific components are pre-anchored within the hydrogel matrix of the device and comprise target-specific capture probes, which are configured to bind a complex formed by the nucleic acid target molecule and the linear RCA probe.

In particular embodiments, said plurality of different pre-defined potential nucleic acid target molecules are selected from the group consisting of DNA from Neisseria meningitidis, Klebsiella pneumoniae, Listeria monocytogenes, Haemophilus influenzae group B, Escherichia coli, Group B Streptococcus, and combinations thereof. Furthermore, in one embodiment, said method allows for simultaneous detection of at least two different pre-defined nucleic acid target molecules from said plurality in different microgel regions of the device.

In another embodiment, the hydrogel microgel deposits of the device are dehydrated after formation and prior to introducing said solution, and rehydrate upon contact with said solution. In yet another embodiment, said detecting comprises fluorescence detection. According to a specific embodiment, said detecting is completed within a time period between 8 minutes and 15 minutes from said introducing said solution. In still a further embodiment, the introducing said polymerase enzyme and nucleotides occurs after said ligating step.

### BRIEF DESCRIPTION OF THE DRAWINGS

The present invention will be understood and appreciated more fully from the following detailed description, taken in conjunction with the drawings in which:

FIGS. 1A and 1B are simplified pictorial assembled and exploded view illustrations of an electrophoretic array assembly constructed and operative in accordance with a preferred embodiment of the invention including a multiplicity of immobilized, mutually spaced and mutually electrically separated microgel regions during rolling-circle amplification (RCA) operation.

FIGS. 2A and 2B are simplified pictorial assembled and exploded view illustrations of the electrophoretic array assembly of FIGS. 1A and 1B including a multiplicity of immobilized, mutually spaced and mutually electrically separated microgel regions in a dehydrated storage operative orientation.

FIGS. 3A-3I are together a simplified illustration of a preferred method of producing the electrophoretic array employed in the embodiment of FIGS. 1A-2B.

FIGS. 4A-4J are simplified illustrations of typical steps in rapid detection of the presence of at least one nucleic acid target molecule in accordance with one embodiment of the present invention.

FIGS. 5A-5J are simplified illustrations of typical steps in rapid detection of the presence of at least one nucleic acid target molecule in accordance with another embodiment of the present invention.

FIGS. 6A-6J are simplified illustrations of typical steps in rapid detection of the presence of at least one nucleic acid target molecule in accordance with yet another embodiment of the present invention.

FIGS. 7A-7J are simplified illustrations of typical steps in rapid detection of the presence of at least one nucleic acid target molecule in accordance with still another preferred embodiment of the present invention.

FIG. 8 is a diagram summarizing the results of Example 1, illustrating the detection of a synthetic DNA target molecule representing Neisseria meningitidis, employing the method of FIGS. 7A-7J.

FIG. 9 is a diagram summarizing the results of Example 2, illustrating the detection of a genomic DNA target molecule extracted from Neisseria meningitidis pathogen spiked into a cerebrospinal fluid clinical sample employing the method of FIGS. 7A-7J.

FIG. 10 is a diagram summarizing the results of Example 3, illustrating the detection of a genomic DNA target molecule extracted from Klebsiella pneumoniae employing the method of FIGS. 7A-7J.

FIG. 11 is a diagram summarizing the results of Example 4, illustrating the simultaneous detection of genomic DNA target molecules extracted from Klebsiella pneumoniae and Listeria monocytogenes employing the method of FIGS. 7A-7J.

FIG. 12 is a diagram summarizing the results of Example 5, illustrating the simultaneous detection of genomic DNA target molecules extracted from Klebsiella pneumoniae and Listeria monocytogenes employing the method of FIGS. 5A-5J.

### DETAILED DESCRIPTION OF THE INVENTION

In the following description, various aspects of the present application will be described. For purposes of explanation, specific details are set forth in order to provide a thorough understanding of the present application. However, it will also be apparent to one skilled in the art that the present application may be practiced without the specific details presented herein. Furthermore, well-known features may be omitted or simplified in order not to obscure the present application.

The terms “comprising,” “comprised of,” “having,” “including,” and their conjugates, mean “including but not limited to.” These terms are open-ended and mean the elements recited, or their equivalent in structure or function, plus any other element or elements which are not recited. They should not be interpreted as being restricted to the means listed thereafter and do not exclude other elements or steps. Thus, the scope of an expression such as “a product comprised of x and z” should not be limited to products composed only of components x and z. Similarly, “a method comprising the steps x and z” should not be limited to methods consisting only of these steps.

The term “consisting of” means “including and limited to.” The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a,” “an,” and “the” include plural references unless the context clearly dictates otherwise. For example, the term “a compound” or “at least one compound” may include a plurality of compounds, including mixtures thereof.

Unless specifically stated, as used herein, the term “about” is understood as within a range of normal tolerance in the art, for example, within two standard deviations of the mean. In some embodiments, “about” means within 10% of the reported numerical value, preferably within 5%, and more preferably within 1%, 0.5%, 0.1%, 0.05%, or 0.01% of the stated value. In other embodiments, “about” can encompass a higher tolerance of variation depending on the experimental technique used or the context of the invention. Said variations of a specified value are understood by the skilled person and are within the context of the present invention. For example, a numerical range of “about 1 to about 5” should be interpreted to include not only the explicitly recited values from about 1 to about 5, but also individual values (e.g., 2, 3, 4) and sub-ranges (e.g., 1-3, 2-4, 3-5) within the indicated range. This principle also applies to ranges reciting only one numerical value as a minimum or a maximum. Unless otherwise clear from context, all numerical values provided herein are modified by the term “about”.

Other similar terms, such as “substantially,” “generally,” “up to,” and the like, are to be construed as modifying a term or value such that it is not an absolute. Such terms will be defined by the circumstances and the terms that they modify as those terms are understood by those skilled in the art. This includes, at very least, the degree of expected experimental error, technical error, and instrumental error for a given experiment, technique, or instrument used to measure a value.

As used herein, the term “and/or” includes any and all combinations of one or more of the associated listed items. It will be understood that when an element is referred to as being “on,” “attached to,” “connected to,” “coupled with,” “contacting,” etc., another element, it can be directly on, attached to, connected to, coupled with, or contacting the other element, or intervening elements may also be present. In contrast, when an element is referred to as being, for example, “directly on,” “directly attached to,” “directly connected to,” “directly coupled with,” or “directly contacting” another element, there are no intervening elements present. References to a structure or feature that is disposed “adjacent” to another feature may have portions that overlap or underlie the adjacent feature.

The present invention provides improved methods, devices, and systems for the rapid, sensitive, and multiplexed detection of nucleic acid target molecules. It addresses the critical need for swift and accurate molecular diagnostics, particularly in scenarios where timely identification of specific nucleic acid sequences, such as those from pathogens or disease biomarkers, can significantly impact outcomes. A central feature of the invention involves the use of electrophoretic arrays incorporating hydrogel microgel deposits that are specifically structured to create optimized localized reaction environments. These structural optimizations facilitate enhanced molecular transport, efficient biochemical reactions such as Rolling Circle Amplification (RCA), and effective concentration of products, leading to significant improvements in detection speed and the ability to detect a plurality of diverse targets.

Unless otherwise specified or clearly required by context, the following terms, as used herein throughout this specification and the appended claims, stand for the definitions provided as follows. The term “nucleic acid target molecule” refers to any nucleic acid sequence, including but not limited to deoxyribonucleic acid (DNA) or ribonucleic acid (RNA), and their natural or synthetic variants or derivatives (such as messenger RNA (mRNA), microRNA (miRNA), complementary DNA (cDNA), genomic DNA, or fragments thereof). The presence, absence, identity, or quantity of such a molecule in a sample is the subject of detection. According to the definition herein, the target molecule contains at least one specific sequence that permits specific recognition and binding by complementary probes or primers as herein described. Examples of nucleic acid target molecules include sequences derived from pathogens such as Neisseria meningitidis, Klebsiella pneumoniae, Listeria monocytogenes, Haemophilus influenzae group B, Escherichia coli, and Group B Streptococcus, among others.

The term “plurality of different pre-defined nucleic acid target molecules” refers to two or more distinct nucleic acid target molecules, each having a unique sequence or originating from a different source (e.g., different pathogens or different genes), which are specifically chosen for detection within a single assay or on a single device.

The term “solution” refers generally to an aqueous liquid medium. As used herein, it may refer to a biological sample suspected of containing the nucleic acid target molecule(s) or to various reagent solutions introduced into the electrophoretic array during the operational steps of the invention, such as solutions containing buffers, enzymes (e.g., ligase, polymerase), nucleotides (dNTPs), primers, probes, or reporters.

“Electrophoretic array,” as defined herein, stands for a device comprising a substrate supporting a plurality of spatially distinct and typically individually addressable electrodes or electrode locations (e.g., carbon electrodes). Such an array is configured to permit the application of electric fields to thereby manipulate, transport, and/or concentrate charged molecules, particularly nucleic acids, within a fluid medium disposed on or within the array. The array typically includes defined microgel regions associated with said electrode locations.

The term “microgel region” signifies a specific, spatially discrete location on the electrophoretic array, typically associated with an individual electrode or a defined set of electrodes. Each microgel region is the site where a hydrogel microgel deposit is immobilized and where the primary biochemical reactions for nucleic acid detection, including amplification, occur. These regions are typically mutually spaced to allow for multiplexed analysis. “Microgel deposit” or “hydrogel microgel deposit,” as used herein, refers to a discrete, localized volume of a hydrogel material that is immobilized at a microgel region on the substrate of the electrophoretic array. It serves as a three-dimensional scaffold or support for pre-anchored biochemical components and provides the localized reaction environment for the steps of the detection method.

“Hydrogel matrix,” as defined herein, stands for the cross-linked polymeric network forming the structural basis or scaffold of the hydrogel microgel deposits. This matrix is typically hydrophilic and capable of absorbing significant amounts of aqueous solution. While a non-limiting example of material described in specific embodiments is cross-linked polyacrylamide formed from acrylamide and N,N′-methylenebisacrylamide (BIS), the invention contemplates that other hydrogel-forming polymers capable of forming the requisite defined porous structure and supporting the biochemical reactions could also be utilized, as the principles of creating porous, functionalized hydrogels are known in the art (e.g., as discussed in U.S. Pat. No. 6,960,298 regarding mesoporous permeation layers). This matrix is typically functionalized for component anchoring, for example, by incorporating an immobilized affinity-binding molecule such as modified streptavidin, which allows for strong and specific binding of biotinylated oligonucleotide components.

The phrase “defined three-dimensional porous structure” or “defined three-dimensional porous hydrogel matrix structure”, as used herein, refers to the interconnected network of void spaces (pores) and polymer chains within the hydrogel matrix. This structure is, in preferred embodiments, a “porogen-derived pore network” (as defined below), meaning its porosity is established, at least in part, through the use of a porogen during hydrogel formation. It is characterized by features such as its pore size distribution, overall porosity, interconnectivity, and degree of cross-linking, which are specifically configured or selected in the present invention to achieve desired functional characteristics, including facilitating transport of nucleic acid molecules up to at least 800 base pairs in length. Preliminary electron microscopy observations indicate that such structures can exhibit pores with diameters ranging, for example, from approximately 350 nm to approximately 5000 nm.

The term “porogen,” as used herein, refers to a substance included in a polymerizable hydrogel solution that, during or after polymerization of the hydrogel matrix, creates or helps define void spaces or pores within the matrix, thereby contributing to its porous structure. A non-limiting example of porogen described herein is APO 10. “Porogen-derived pore network,” as defined herein, refers to the inter-connected system of pores or void spaces within the hydrogel matrix that results from the inclusion of a porogen in the polymerizable hydrogel solution during the formation of the hydrogel.

“Optimized for rapid molecular transport,” as used herein when referring to the porous structure of the hydrogel matrix, means that the structure, particularly when a porogen-derived pore network is present, is configured to facilitate efficient diffusion and/or electric-field driven migration of relevant biomolecules (such as target nucleic acids, including fragments up to at least 800 base pairs in length, primers, probes, enzymes, and nucleotides) into and within said hydrogel matrix, at a rate that contributes to the overall sub-20-minute detection timeframe of the method, as compared to a non-optimized or less porous gel structure.

“Localized reaction environment” stands for the confined three-dimensional volume provided by the hydrogel microgel deposit, within which biochemical reactions such as hybridization, ligation, and amplification are intended to predominantly occur. This environment facilitates increased local concentrations of reactants and reaction products.

“Pre-anchored components” refers to one or more types of molecules, typically oligonucleotides such as capture probes, primers, or RCA probes, that are immobilized or attached within the hydrogel microgel deposit prior to the introduction of the sample solution containing the nucleic acid target molecule(s). “Affinity-binding molecule,” as defined herein, stands for a molecule immobilized within the hydrogel matrix that possesses a specific binding affinity for another molecule or a molecular tag, thereby facilitating the anchoring or immobilisation of said other molecule (e.g., a biotinylated oligonucleotide) to the hydrogel matrix. A non-limiting example of the affinity-binding molecule described herein is modified streptavidin.

“Target-specific components,” as defined herein, are pre-anchored components that possess a sequence or binding capability that is specific for one of the pre-selected nucleic acid target molecules or for a derivative thereof (e.g., an RCA probe specific for the target). “Target-specific capture probe” signifies an oligonucleotide or other molecular entity that is pre-anchored within the hydrogel microgel deposit and is designed to specifically hybridize or otherwise bind to a nucleic acid target molecule, or to a complex comprising the target molecule (such as a target-RCA probe complex), thereby serving to immobilize or concentrate said target or complex within the microgel region.

“Primer,” as used throughout this specification, refers to a short oligonucleotide sequence, typically DNA, designed to anneal or hybridize to a specific complementary sequence on a template nucleic acid strand. This hybridization provides a starting point (typically a free 3′-hydroxyl group) for a polymerase enzyme to initiate the synthesis of a new nucleic acid strand complementary to the template. “Forward primer” generally refers to a primer that initiates DNA synthesis in one direction along a template strand. “Reverse primer” generally refers to a primer that initiates DNA synthesis on the complementary strand or in the reverse direction relative to the forward primer, often used in producing double-stranded DNA or in exponential amplification schemes.

A “barcode sequence,” as used herein, refers to a specific, typically artificial, oligonucleotide sequence that is incorporated into a larger nucleic acid molecule, such as a linear RCA probe. Its function is not to bind to the primary nucleic acid target molecule, but rather to serve as a unique identifier or “tag” for the probe it is part of. In the context of the present invention, a barcode sequence on a linear RCA probe acts as a specific hybridization site for a corresponding pre-anchored, barcode-specific primer. This mechanism localizes the correct RCA probe to a designated microgel region, which is a key strategy for enabling highly specific, multiplexed analysis on the array.

“Polymerizable hydrogel spotting solution,” as used herein, refers to a liquid mixture containing monomers capable of forming a cross-linked polymer (e.g., acrylamide and BIS), typically a porogen, an affinity-binding molecule or its precursors, and a polymerization initiator, which solution is spotted onto a substrate and subsequently cured to form a hydrogel microgel deposit. “Curing,” as defined herein in the context of forming hydrogel microgel deposits, refers to the process of inducing polymerization and cross-linking of the components in the polymerizable hydrogel spotting solution to form a stable hydrogel matrix. According to an embodiment described herein, this may include UV curing in the presence of a photoinitiator.

The term “photoinitiator,” as used herein, stands for a compound that, upon absorption of light (typically UV light), generates reactive species that initiate polymerization of monomers to form a polymer. A non-limiting example of the photo-initiator used in embodiments described herein is Darocur 4265. Any other suitable photoinitiator can be used as well.

“Rolling Circle Amplification (RCA),” as defined herein, refers to an isothermal nucleic acid amplification method wherein a DNA polymerase extends a primer hybridized to a circular DNA template, with the polymerase possessing strand displacement activity, thereby continuously synthesizing a long single-stranded DNA molecule composed of tandem repeats of the sequence complementary to the circular template.

“Linear RCA probe” stands for a single-stranded oligonucleotide probe that has distinct 5′ and 3′ ends and is designed to hybridize, typically via its end regions, to a nucleic acid target molecule at adjacent or nearby positions. Upon such hybridization, its ends are brought into proximity, making it a substrate for enzymatic ligation to form a circular molecule. The linear RCA probe also typically contains sequences, which are complementary to one or more primers for RCA.

“Circular RCA probe” or “circular RCA probe template” refers to a single-stranded, covalently closed circular nucleic acid molecule that serves as the template for the RCA reaction. It is typically formed by the ligation of a linear RCA probe that has hybridized to its specific target. “Ligation” or “ligating” signifies the enzymatic reaction that forms a phosphodiester bond to join two nucleic acid ends, for example, the 5′ phosphate end and the 3′ hydroxyl end of a linear RCA probe when appropriately positioned on a target molecule, thereby circularizing the probe. This reaction is typically catalysed by a DNA ligase enzyme.

“Complex” or “ligation-competent complex,” according to the definition herein, refers to an assembly of molecules formed, at a minimum, by the specific hybridization of a nucleic acid target molecule with a linear RCA probe such that the 5′ and 3′ ends of the linear RCA probe are juxtaposed correctly for enzymatic ligation. The complex may also include one or more primers hybridized to the linear RCA probe.

“Polymerase enzyme” catalyses the synthesis of polynucleotide chains from deoxyribonucleotide triphosphates (dNTPs) or ribonucleotide triphosphates (NTPs), using an existing nucleic acid strand as a template. For RCA, a polymerase with strand-displacement activity (e.g., Bst polymerase, Phi29 polymerase) is typically used.

“Amplifying” or “amplification” refers to the process of enzymatically producing multiple copies of a specific nucleic acid sequence or its complement. As described herein, this primarily refers to RCA. “Amplicon(s)” refers to the nucleic acid molecules that are the products of an amplification reaction. In the context of RCA, this term typically denotes the long, single-stranded DNA molecules containing tandem repeats of the sequence complementary to the circular RCA probe template, and may also include any secondary products if further amplification steps occur.

“Electric field(s)” signifies the region of influence created by a voltage difference applied between electrodes of the electrophoretic array, which exerts a force on charged molecules (such as nucleic acids, which are typically negatively charged) present within the solution or microgel, thereby inducing their movement (electrophoresis or electroosmosis) or influencing their orientation or concentration.

“Reporter(s),” as used herein, stands for a molecule or molecular complex used to detect the presence of amplicons. Typically, a reporter comprises a detectable label (e.g., a fluorophore, an enzyme, a nanoparticle) and a component that allows it to specifically or preferentially associate with the amplicons (e.g., a complementary oligonucleotide probe).

“Detecting” or “detection” refers to the process of identifying and/or quantifying a signal generated by the reporter molecules associated with the amplicons that are concentrated within a microgel region, which serves as an indication of the presence (and potentially quantity) of the specific nucleic acid target molecule in the original sample. The term “multiplex detection” refers to the simultaneous or parallel detection of two or more different pre-defined nucleic acid target molecules in the same sample using a single device or assay run, typically by employing different target-specific components in spatially discrete microgel regions.

“Room-temperature shelf-storable” describes the characteristic of an electrophoretic array device, particularly when its microgel deposits containing pre-anchored components are in a dehydrated state, to be stored for extended periods at ambient room temperatures (e.g. approximately 15-30° C.) without requiring refrigeration or freezing, while substantially maintaining its functional integrity for subsequent use in the detection method.

Identifying specific nucleic acid target molecules from complex biological samples often presents a significant challenge, akin to finding a needle in a haystack. Imagine, for instance, a clinical setting where a patient is suspected of having a serious infection, such as meningitis. The patient's sample, which could be cerebrospinal fluid (CSF), blood, or another bodily fluid, contains a vast and complex mixture of genetic material. This includes the patient's own genomic DNA, RNA, and potentially nucleic acids from commensal microorganisms. Amidst this overwhelming background, the specific DNA or RNA signature of the causative pathogen (the “needle”) might be present in very low quantities.

Finding this specific pathogenic nucleic acid quickly and reliably is akin to searching for a few unique, critically important books scattered throughout a massive, disorganized library. Traditional molecular diagnostic methods, while often sensitive, can be like a manual, shelf-by-shelf search of this library—they eventually find the book, but the sequential nature of the diagnostic process can take many hours or even days. In many medical situations, such a delay is unacceptable, as effective and life-saving treatment often depends on rapid identification of the specific pathogen or biomarker. There is a persistent need for methods that can drastically reduce this search time without sacrificing accuracy or the ability to identify multiple different “books” (targets) simultaneously. The inventive solution disclosed in the present invention is an electrically-charged “library” with multiple super-efficient “search stations”, that is optimized structured microgels.

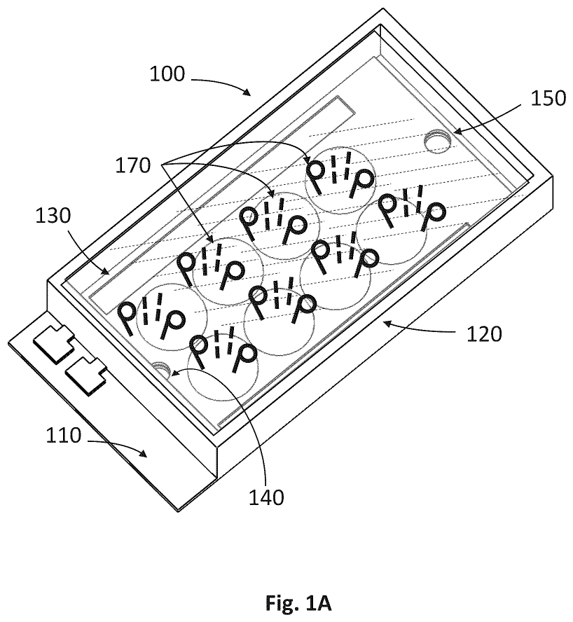

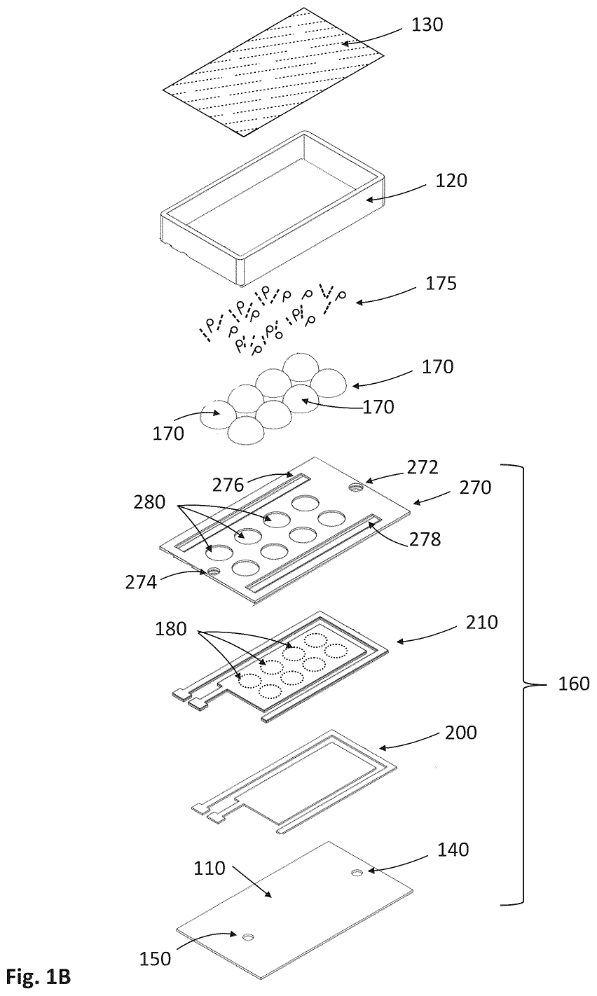

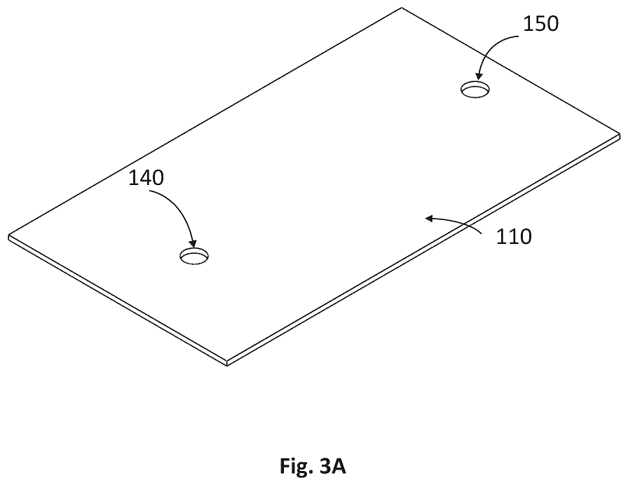

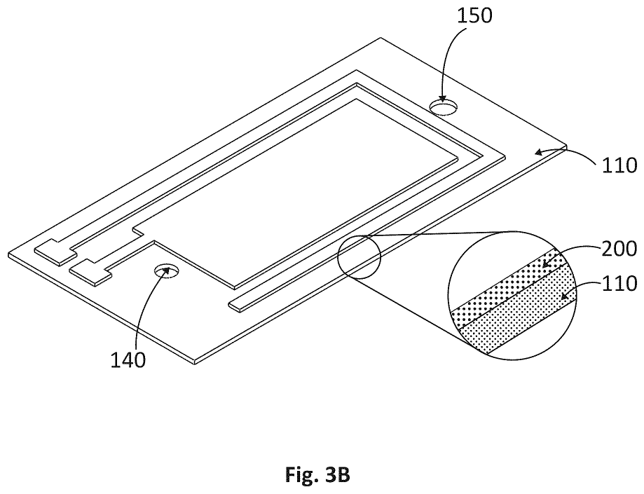

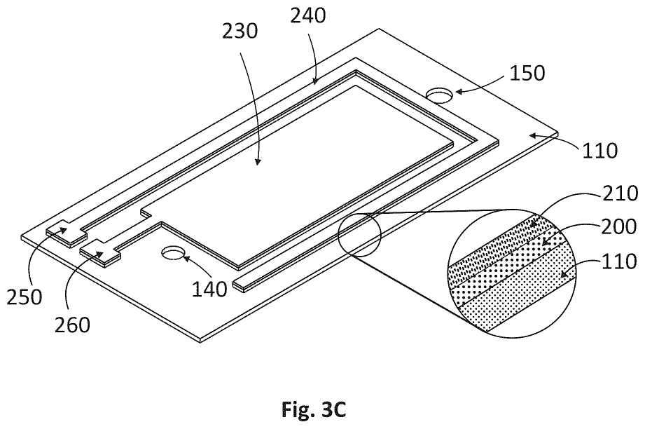

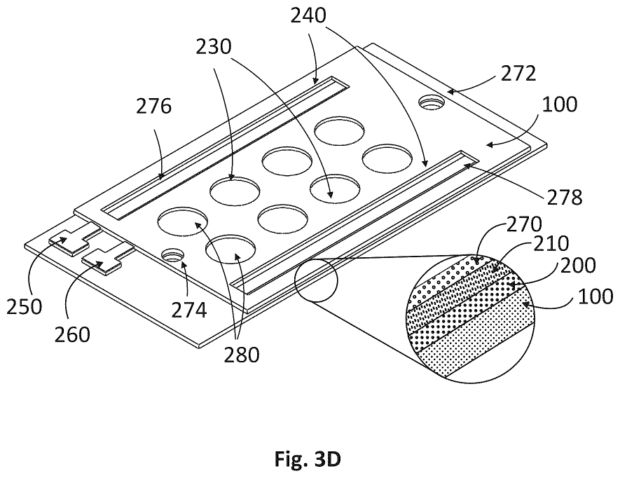

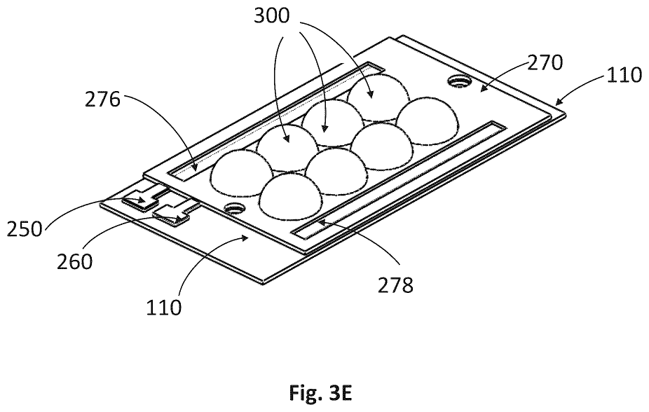

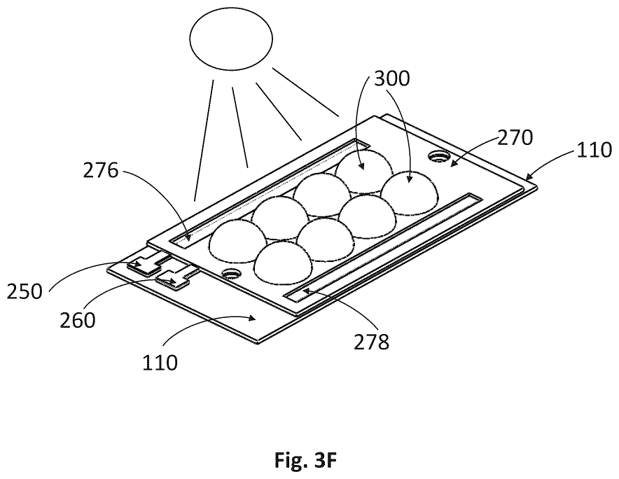

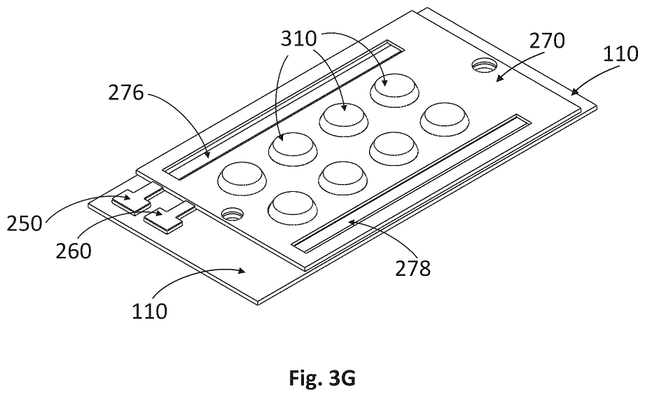

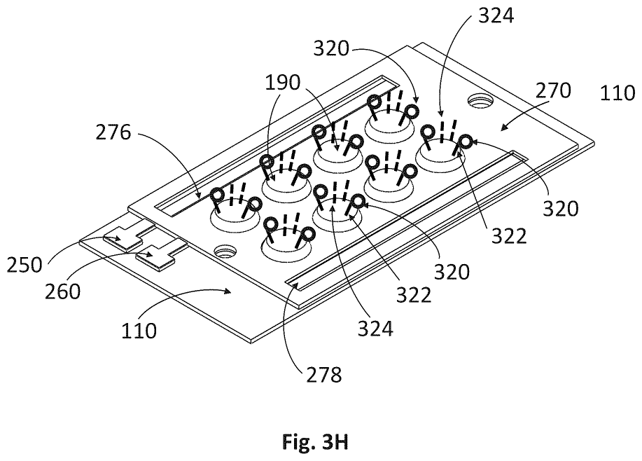

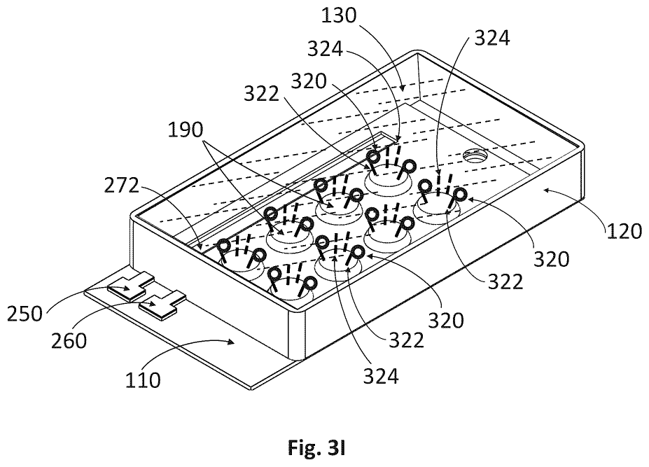

The present invention offers a dramatically faster and more efficient approach to finding these specific “books”—the nucleic acid target molecules. At its core, the invention utilizes a specially designed device, an electrophoretic array, which can be thought of as a highly organized, modern library designed for speed and precizion, equipped with exceptionally skilled “librarians” or “search stations” in the form of structured microgels. The platform for this rapid detection is an electrophoretic array, as generally illustrated in various configurations in FIGS. 1A and 1B and with aspects of its construction process detailed in the parent application, for instance, in procedures analogous to those shown in its FIGS. 3A-3I.

The general architecture and operational principles of suitable electrophoretic arrays and active electronic matrix devices are also described in U.S. Patents such as U.S. Pat. Nos. 5,605,662, 6,238,624, 6,403,367, 6,524,517, 6,960,298, 7,101,661, 7,601,493, and 8,288,155, the disclosures of which are incorporated herein by reference in their entireties for their teachings on array design, fabrication, and material specifications.

As illustrated in FIGS. 1A (the assembled view), showing an electrophoretic array assembly (100), and FIG. 1B (the exploded view), the array typically comprises a substrate (110), which forms the base of the “library”. Formed on this substrate is an electrophoretic array (160), which includes a plurality of precisely positioned electrodes, e.g., working electrodes at discrete electrode locations (180) and a counter electrode (240), as detailed for an exemplary embodiment in FIGS. 3B-3D. These electrodes, often carbon electrodes as used in preferred embodiments detailed in Example 6 (the hydrogel preparation protocol), are crucial for the “express delivery system” described later.

The array assembly often includes a peripheral wall structure (120) and a window (130) to define a chamber or flow cell for containing the sample solution. Access for introducing the sample solution and other reagents can be provided by solution ingress (140) and egress (150) apertures.

A key inventive feature of the present disclosure lies in the nature of the microgel deposits (170) immobilized at the discrete electrode locations (180) on the array. These are not mere passive supports but are highly specialized hydrogel microgel deposits, each characterized by a defined three-dimensional porous hydrogel matrix structure and equipped with molecular components for specific identification of pre-defined potential nucleic acid target molecules. Think of these as the “super-librarians”, or highly efficient, pre-programmed search stations within our electric library. Each microgel deposit defines a microgel region, a localized, optimized reaction hub for all subsequent biochemical reactions.

The defined three-dimensional porous hydrogel matrix structure of the microgel deposits, which is specifically configured to provide a localized reaction environment and is optimized for rapid molecular transport of nucleic acid molecules up to at least 800 base pairs in length, is critical to the enhanced performance of the present invention. Achieving this optimized structure involves specific steps in the formation of the hydrogel microgel deposits on the electrophoretic array, as exemplified in Example 6 (the hydrogel preparation protocol).

In preferred embodiments, the hydrogel microgel deposit is formed by spotting a polymerizable hydrogel spotting solution onto a microgel region and subsequently curing said solution. This polymerizable hydrogel spotting solution typically comprises several key components: monomers for forming said cross-linked polymer, a porogen for forming the porogen-derived pore network, an affinity-binding molecule or its precursors (e.g., modified streptavidin), and a polymerization initiator. The curing step, when a photoinitiator is used, typically comprises UV curing, for instance, using a hand-held UV device for a defined period (e.g., 10 seconds) as described in Example 6 (the hydrogel preparation protocol).

The inclusion of a porogen like APO 10 during polymerization is a key factor in creating the porogen-derived pore network defining interconnected void spaces within said matrix. This network results in a porous structure that is crucial for the device's function. While precise statistical characterization of average pore diameter across all embodiments may vary, preliminary electron microscopy observations of hydrogels prepared using such porogen-inclusive protocols have indicated the presence of pores with diameters that can range, for example, from approximately 350 nm to approximately 5000 nm. Such a range of interconnected void spaces is consistent with, and enables, the matrix's capability for rapid molecular transport of nucleic acid molecules up to at least 800 base pairs in length, allowing analytes (such as the nucleic acid “books” including both target and non-target DNA molecules) and reagents (“search tools” like enzymes and building blocks) to efficiently move into and within the matrix. This specific optimization also involves controlling the degree of cross-linking, influenced by the monomer/BIS ratio, which also impacts the mechanical stability of the microgel.

The defined three-dimensional porous structure of these microgels is critical. Instead of a dense, impermeable blob, imagine each microgel as a microscopic sponge or a finely tuned 3D lattice. This structure facilitates not only transport but also provides a high effective surface area within the localized reaction environment for efficient interaction between solution-phase molecules and pre-anchored components.

Each “super-librarian” microgel is highly specialized, containing target-specific components pre-anchored within its hydrogel matrix, typically via the immobilized affinity-binding molecule (e.g., biotinylated oligonucleotides anchored via modified streptavidin). This pre-anchoring means each station is ready to look for a specific type of “book” (a particular nucleic acid target molecule).

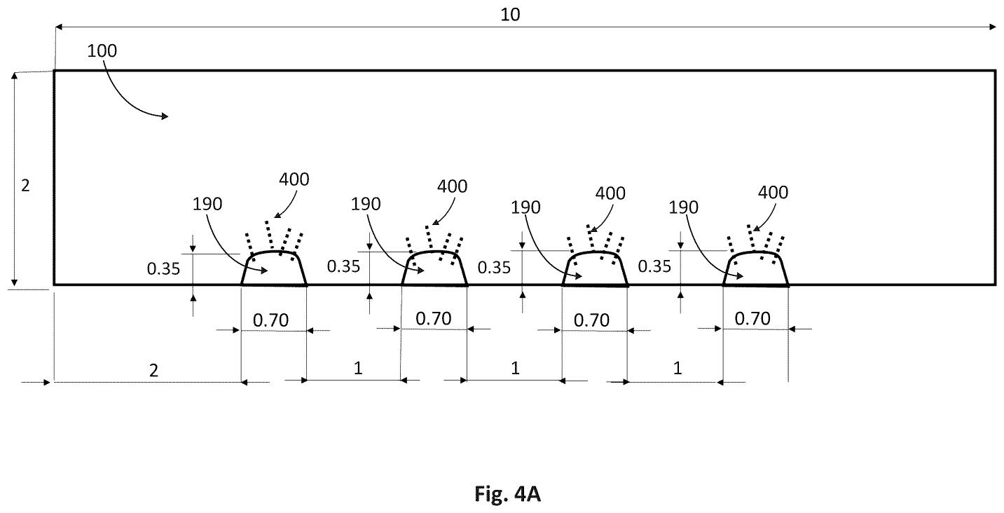

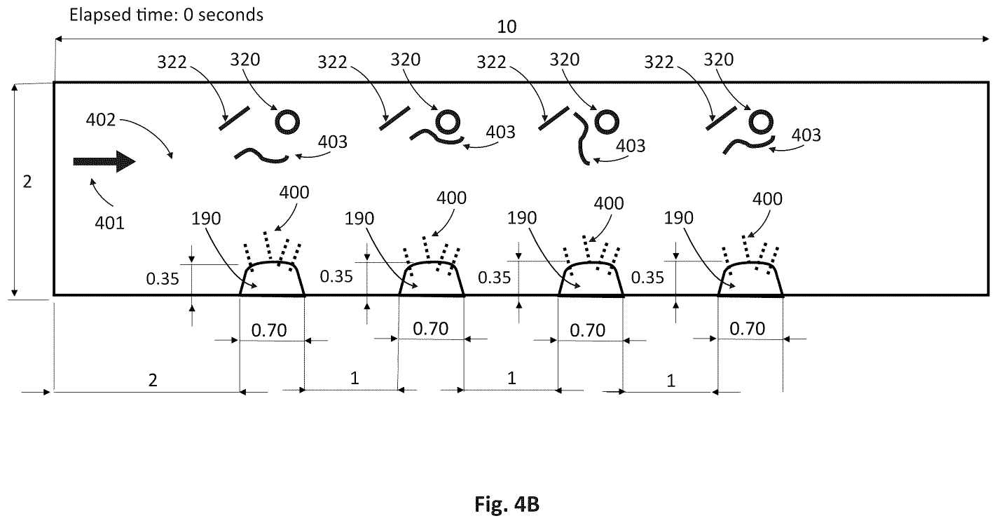

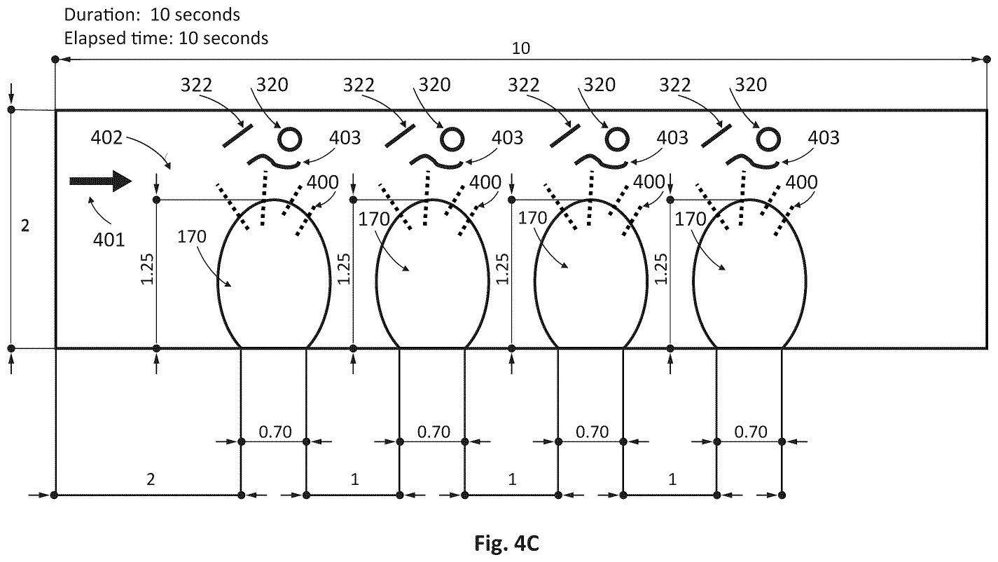

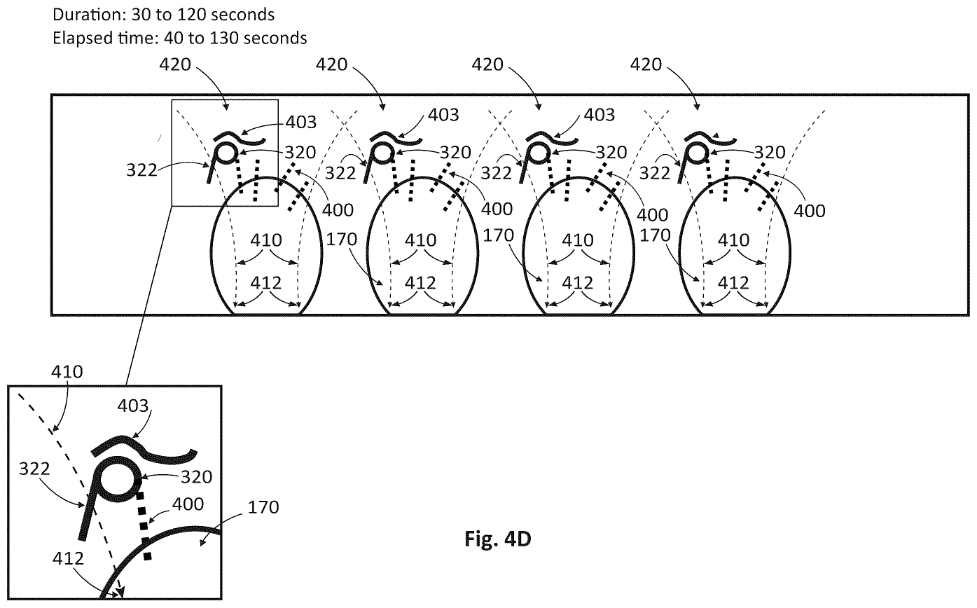

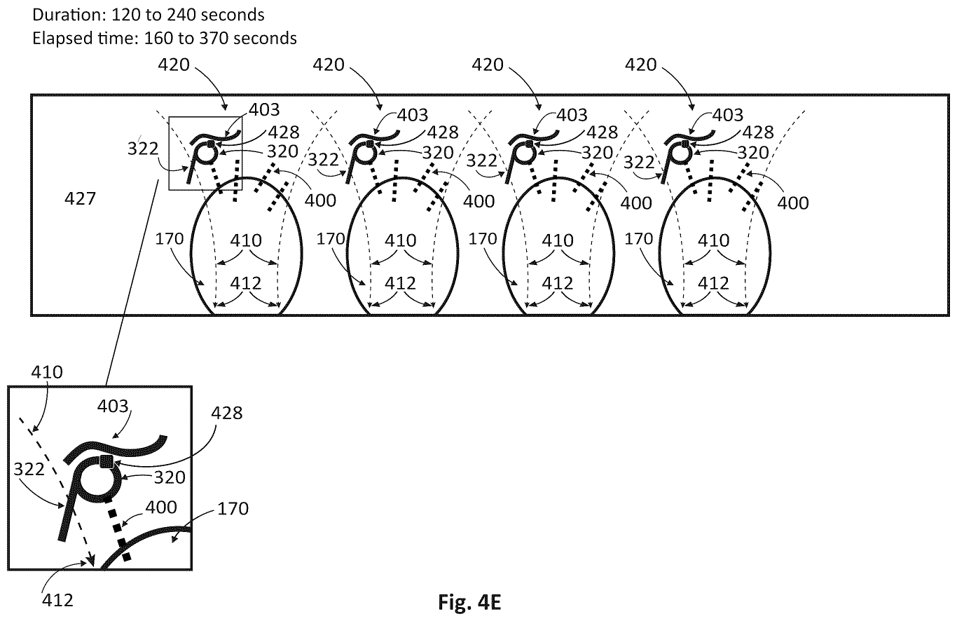

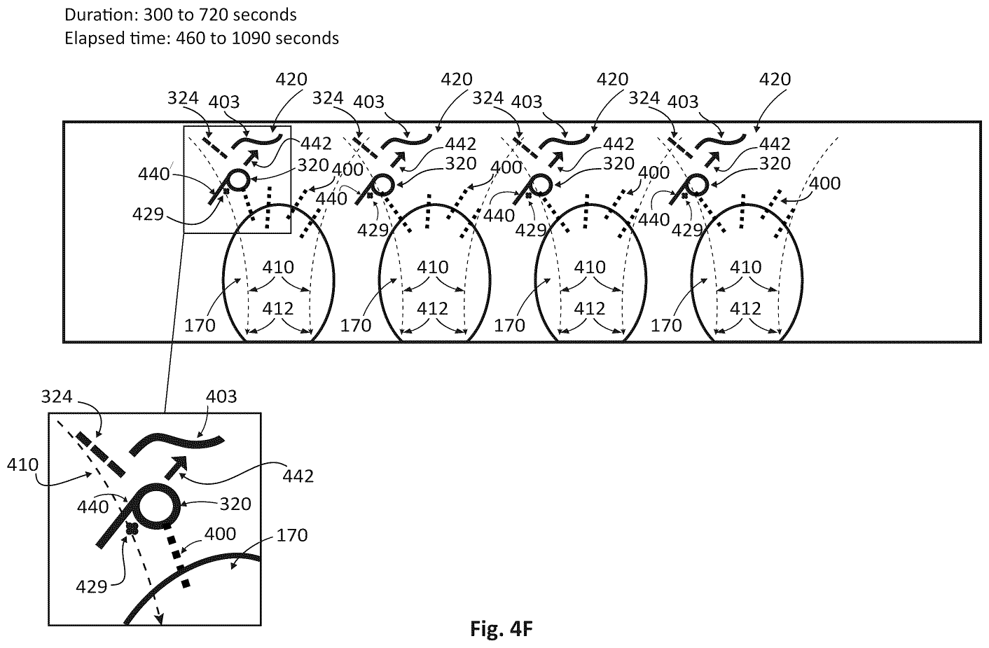

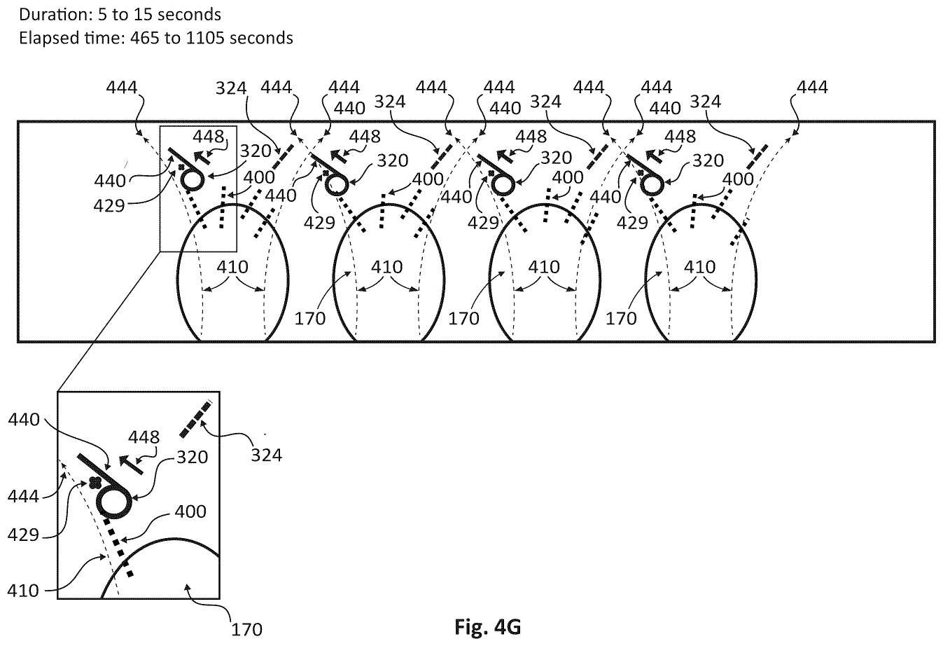

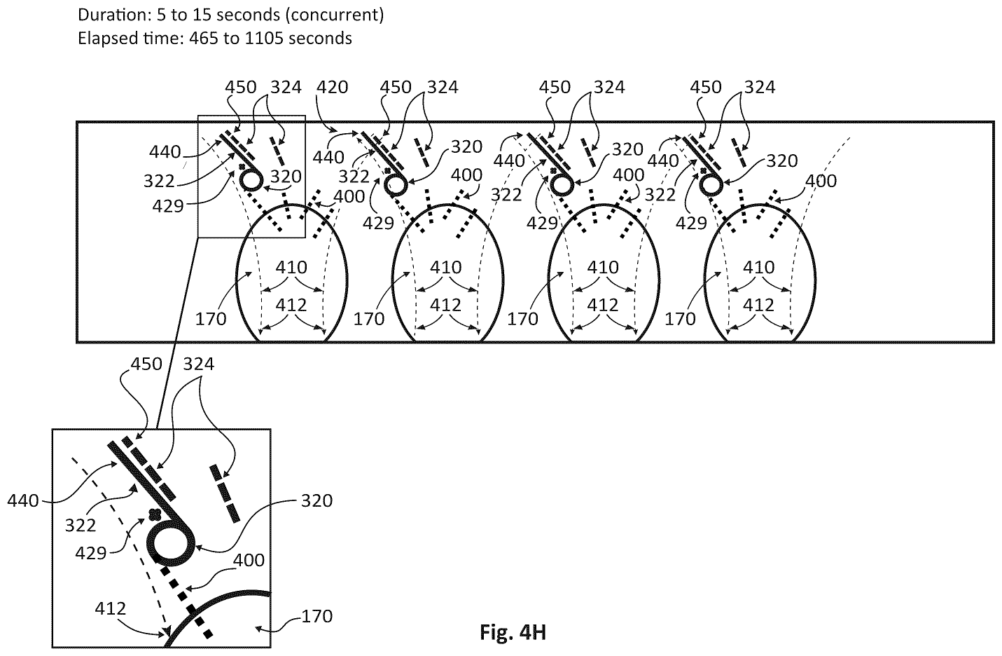

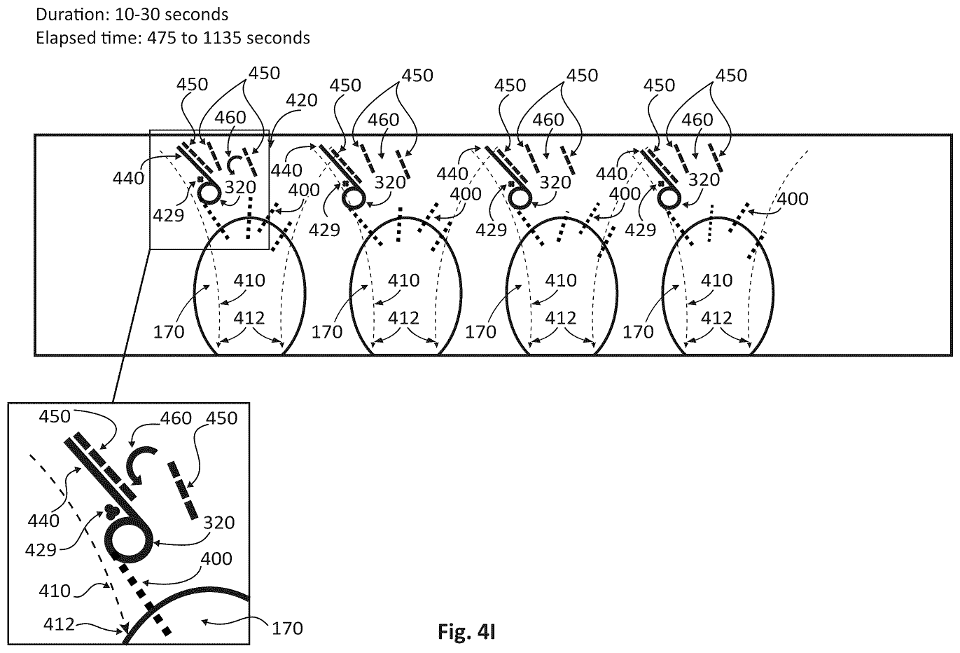

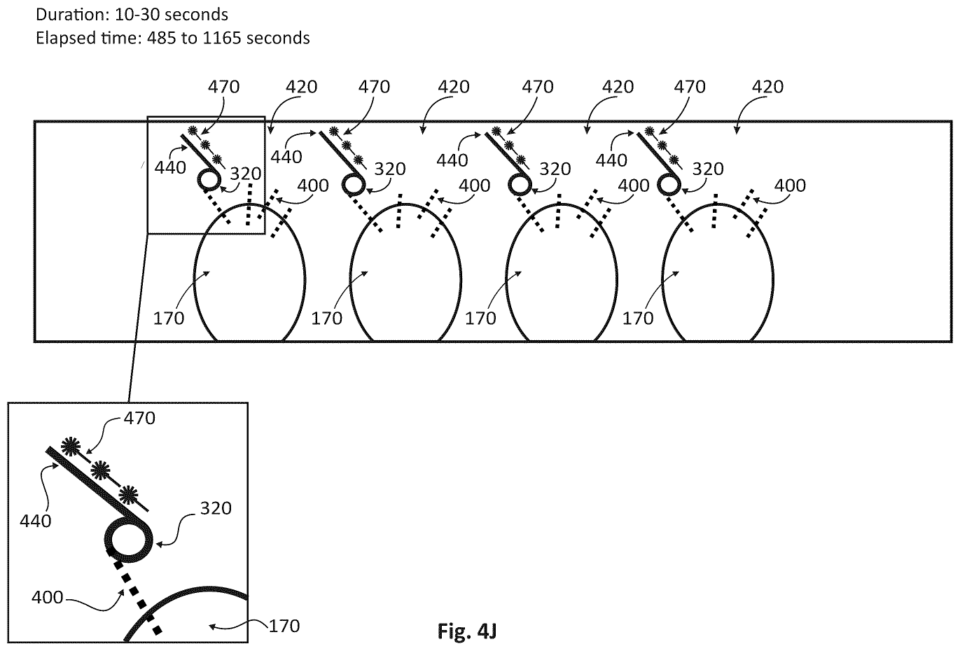

In some configurations, these pre-anchored components are target-specific capture probes. These act like a librarian holding up a sign with the exact title or unique identifier of the book they are searching for. This configuration is generally illustrated in the operational steps of FIGS. 4A-4J, where capture probes (400 in FIG. 4A) are bound to the microgel deposits (190).

In other configurations, the pre-anchored components are primers designed to be specific not to the nucleic acid target molecule itself, but to a unique barcode sequence integrated into the linear RCA probe for that specific target. This configuration is highly effective for multiplexing, where each microgel designated for a particular target is pre-anchored with primers specific to the barcode of that target's corresponding linear RCA probe. This ensures that when a solution containing various targets and their corresponding barcoded RCA probes is introduced, each RCA probe (once bound to its target) is localized to its designated microgel region via specific hybridization between its barcode and the anchored primer. This technically sound approach avoids potential competition between the anchored primer and the RCA probe for binding to the same nucleic acid target. This is like the librarian having the first few sentences of a chapter ready to instantly match an incoming book.

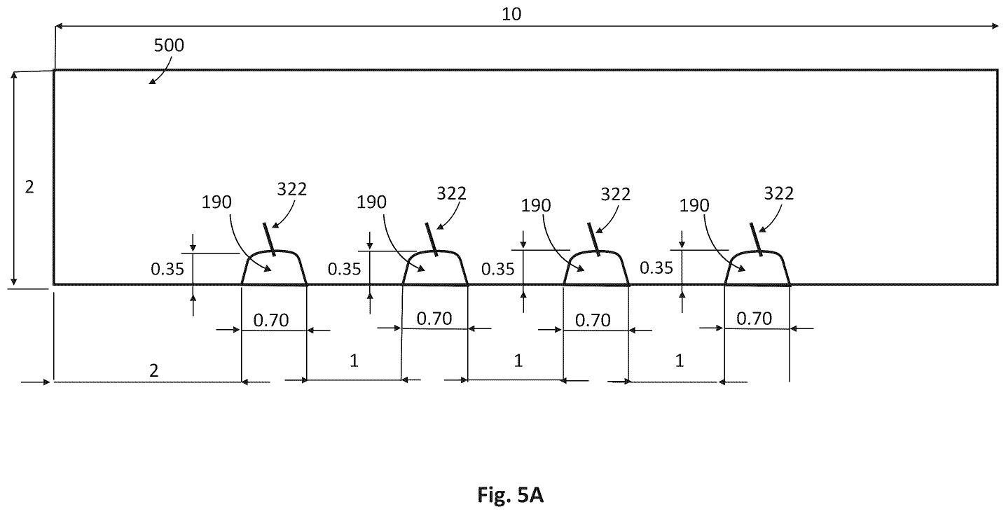

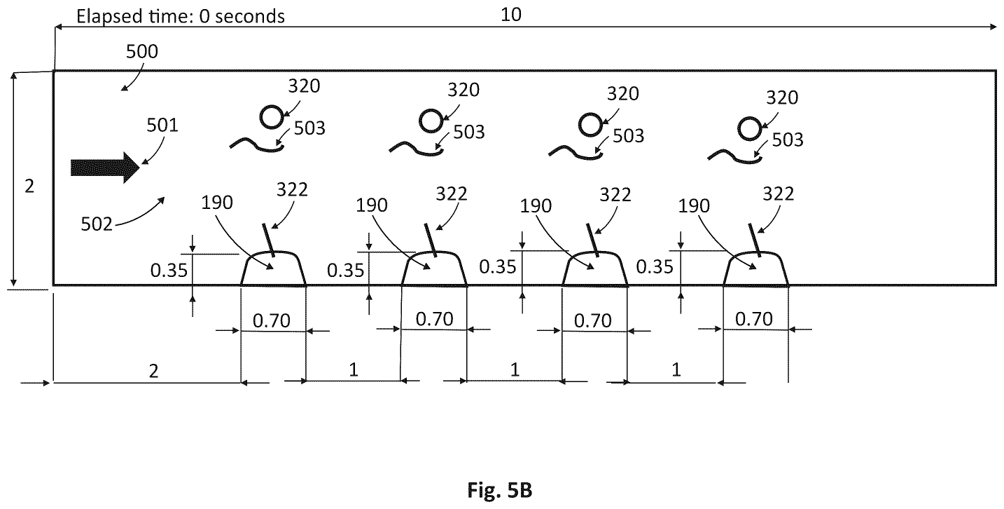

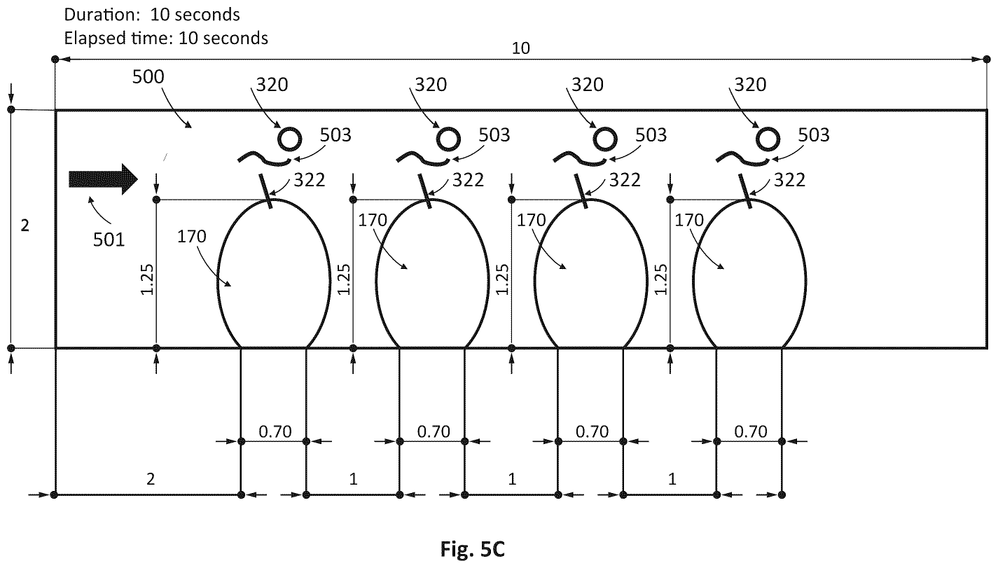

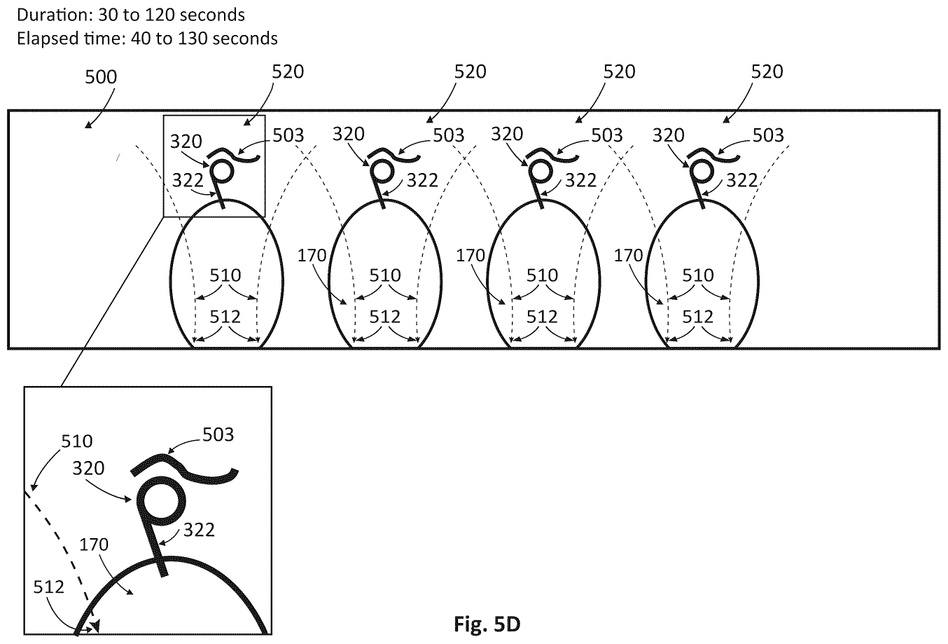

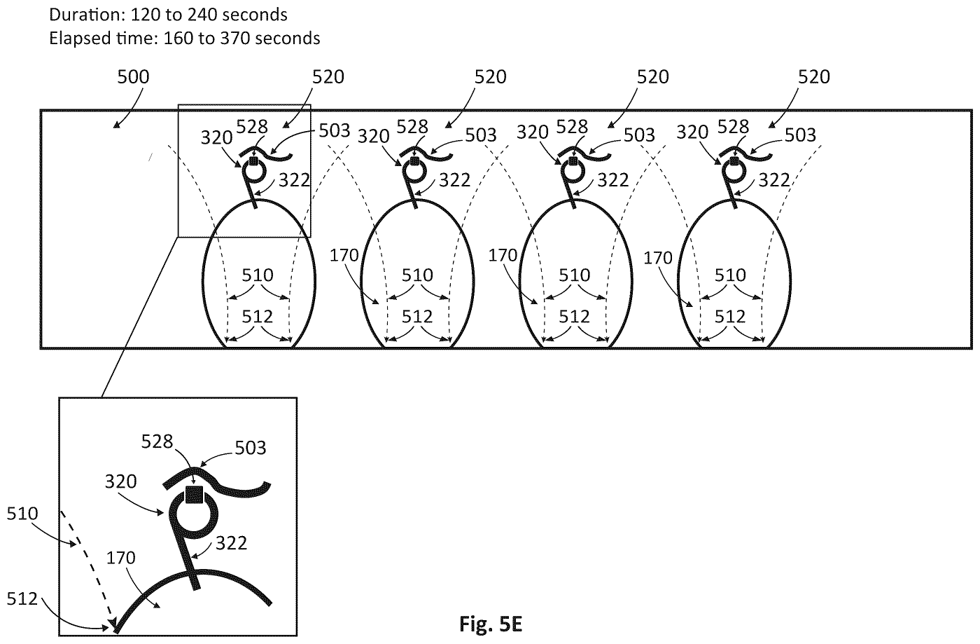

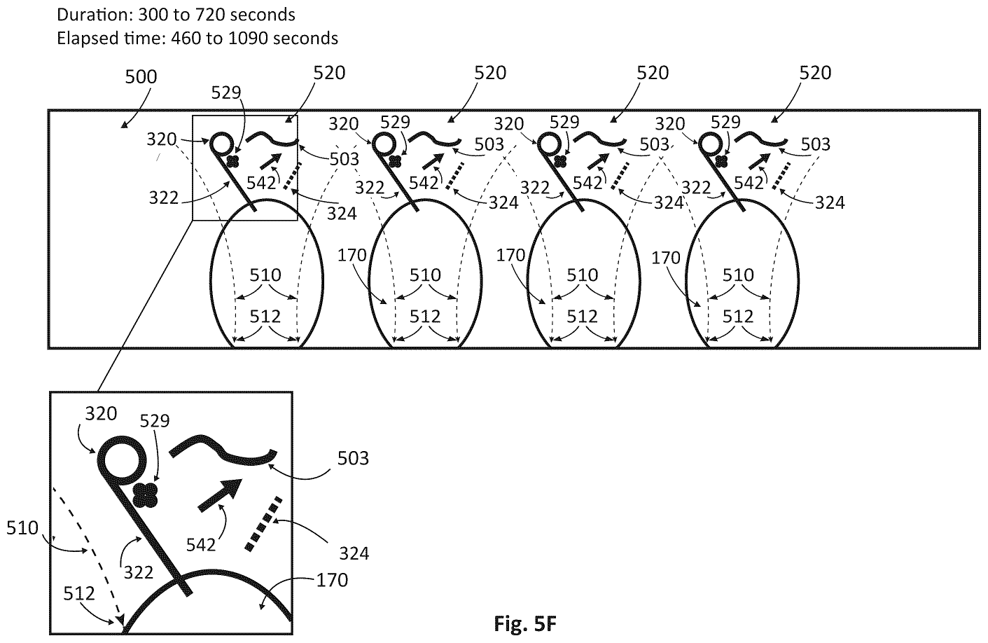

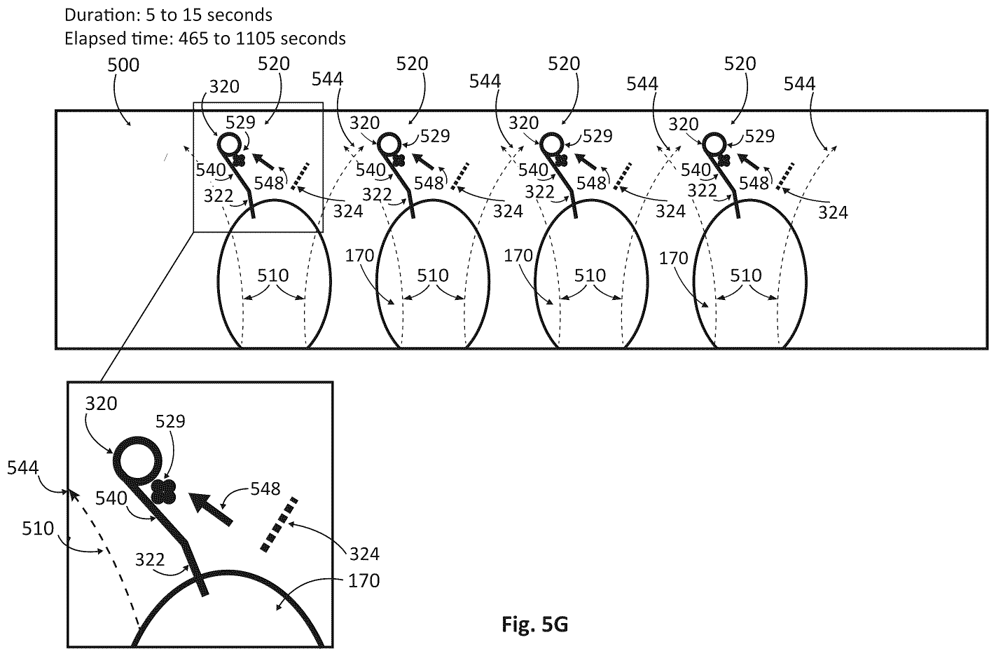

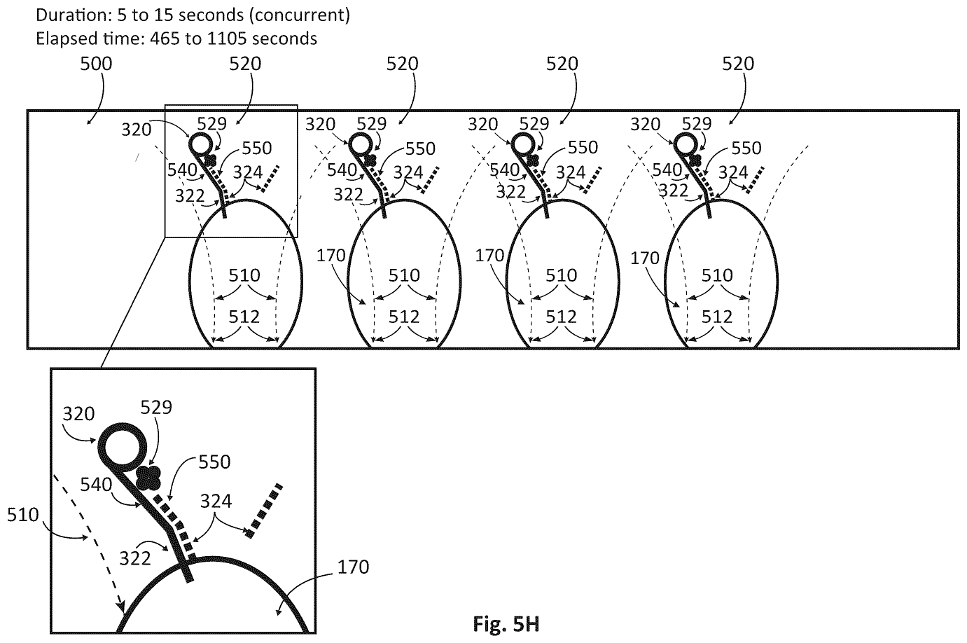

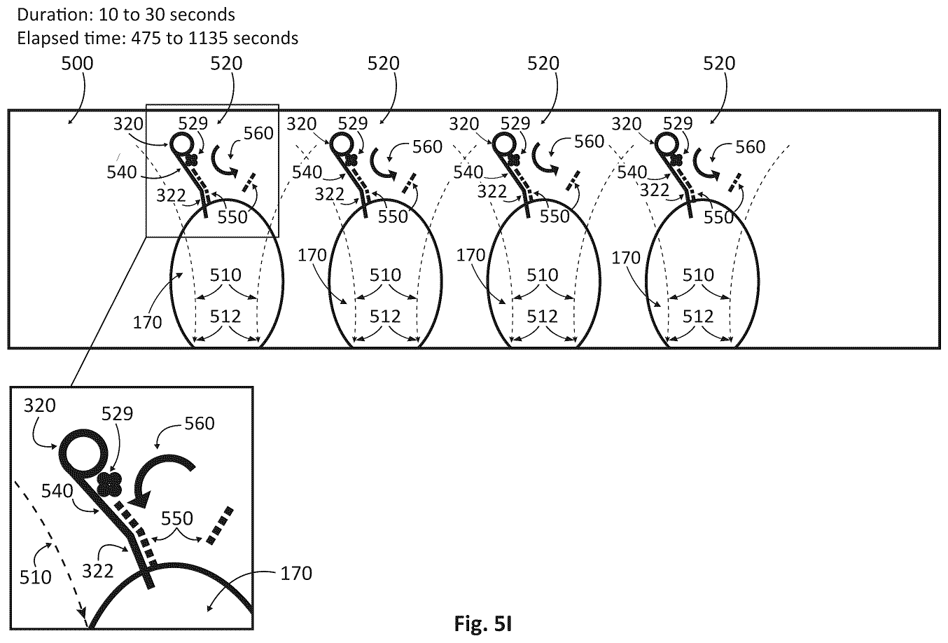

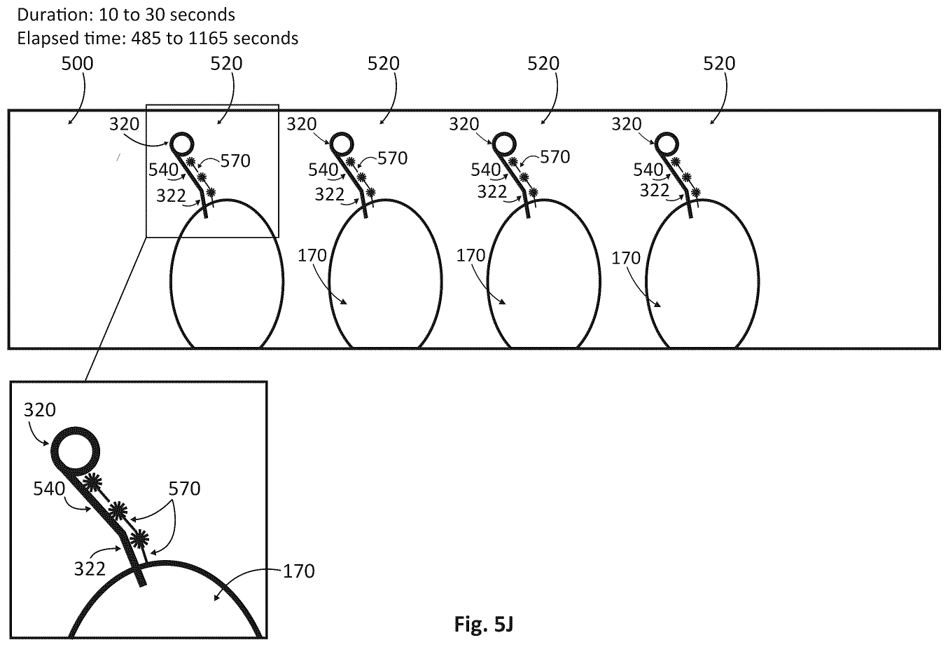

This scenario is depicted generally in FIGS. 5A-5J. In this embodiment, the pre-anchored components are forward primers (322 in FIG. 5A). These primers are specific to a unique barcode sequence present on the corresponding linear RCA probe (320), which is introduced in solution along with the nucleic acid target molecule (503). As shown conceptually in FIG. 5D, the RCA probe first binds to its target molecule in solution, and this entire complex is then captured and localized to the correct microgel region (170) by the specific hybridization of the anchored primer (322) to the barcode sequence on the RCA probe (320).

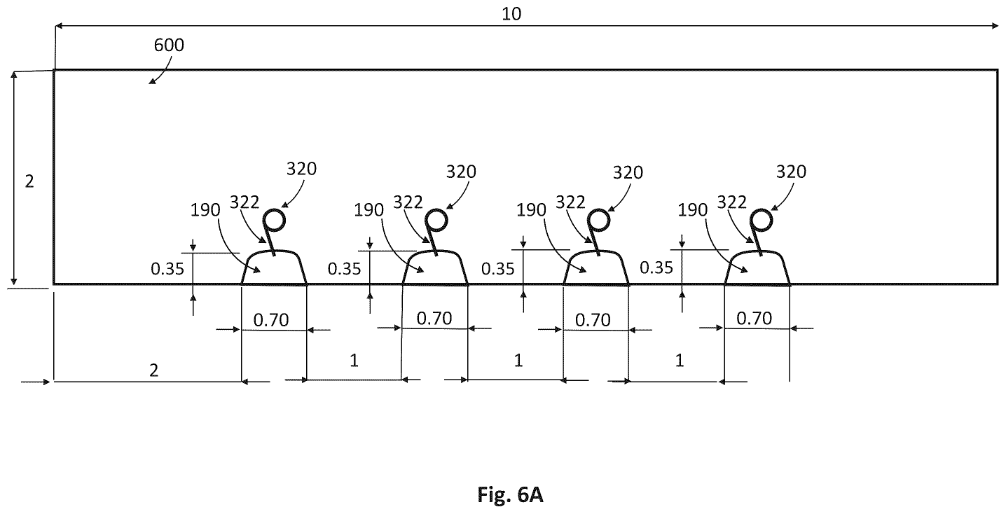

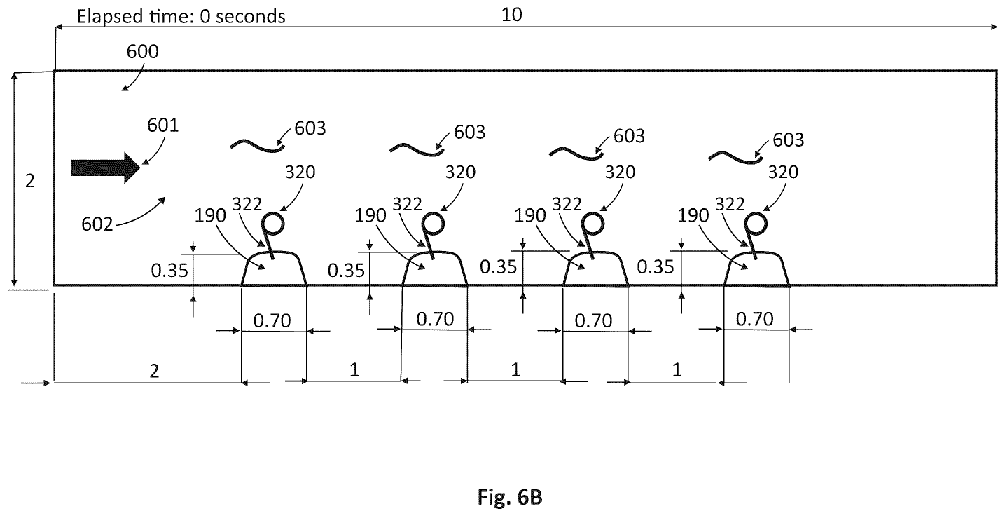

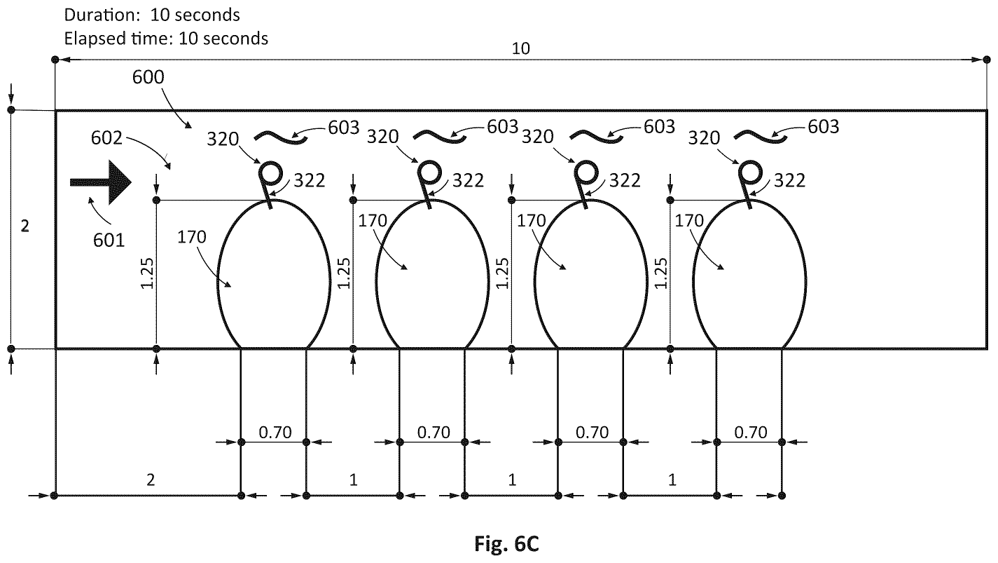

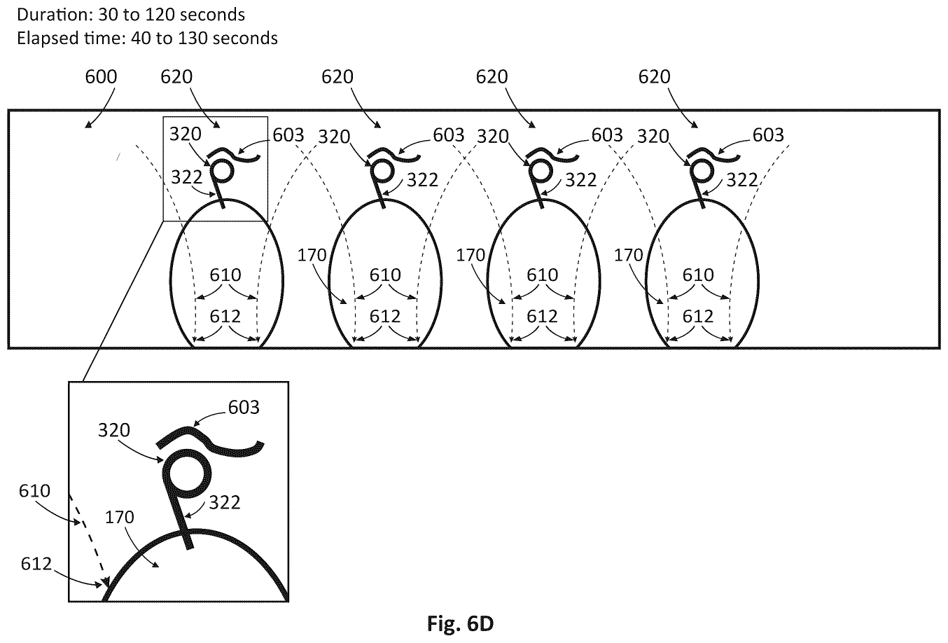

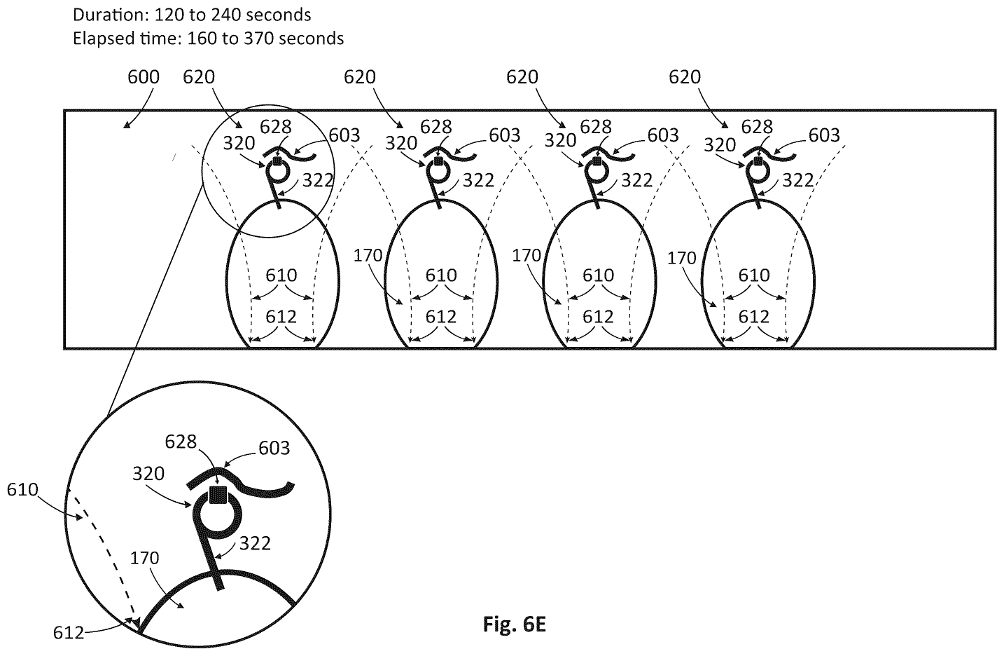

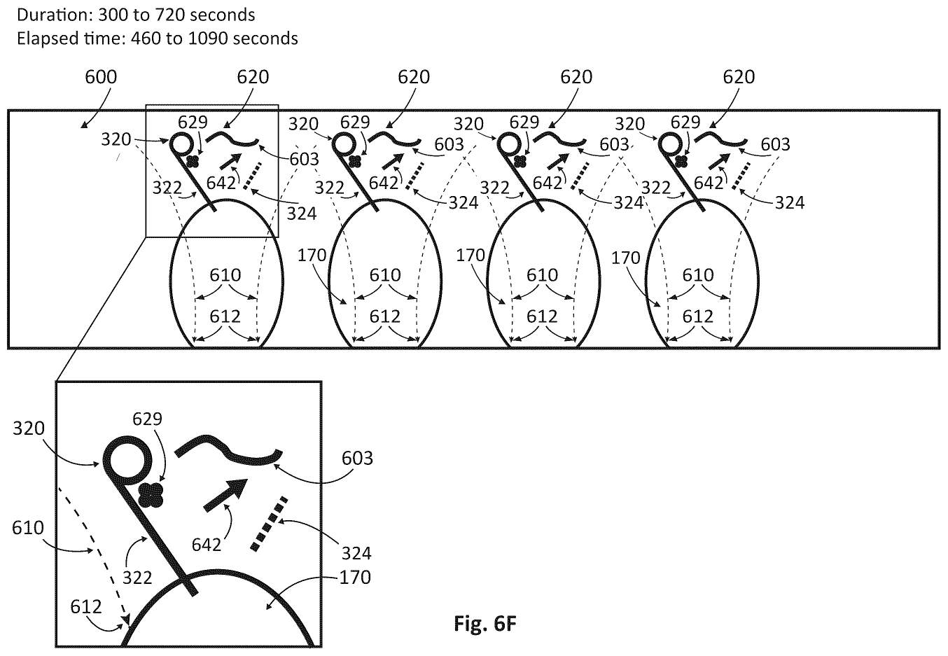

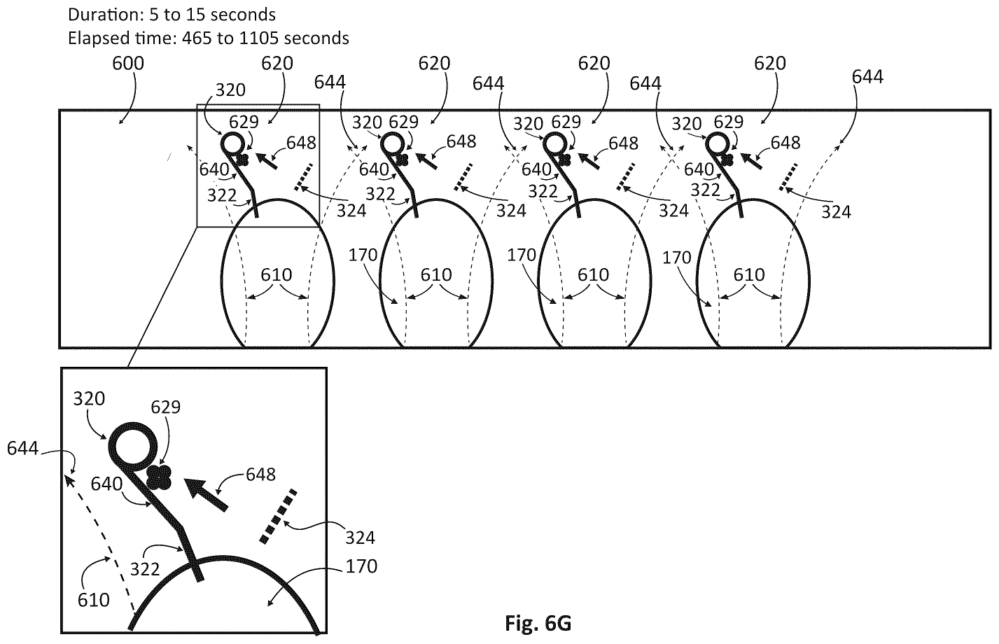

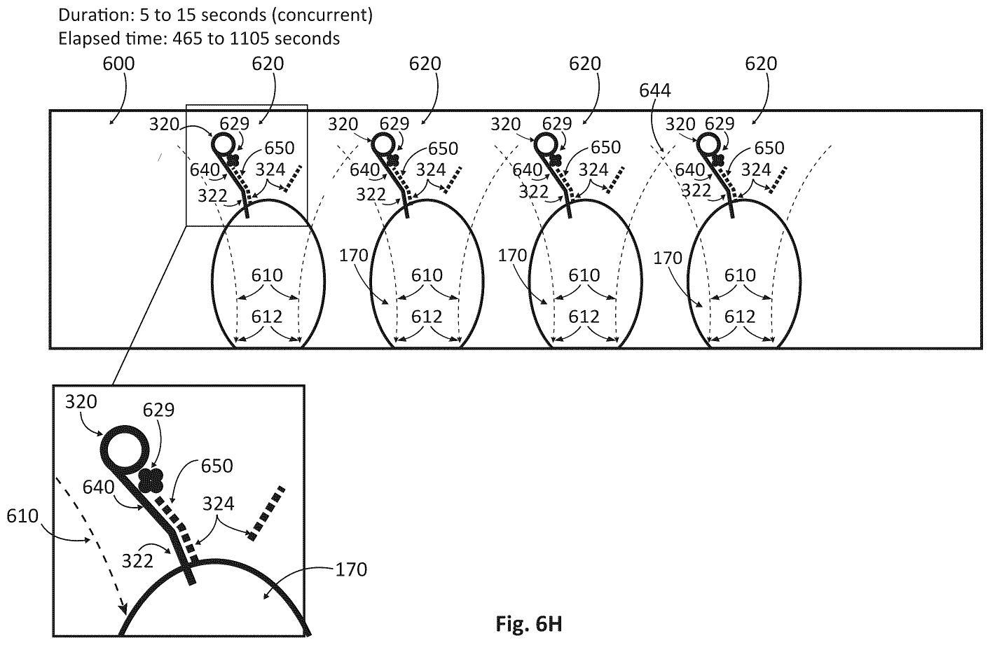

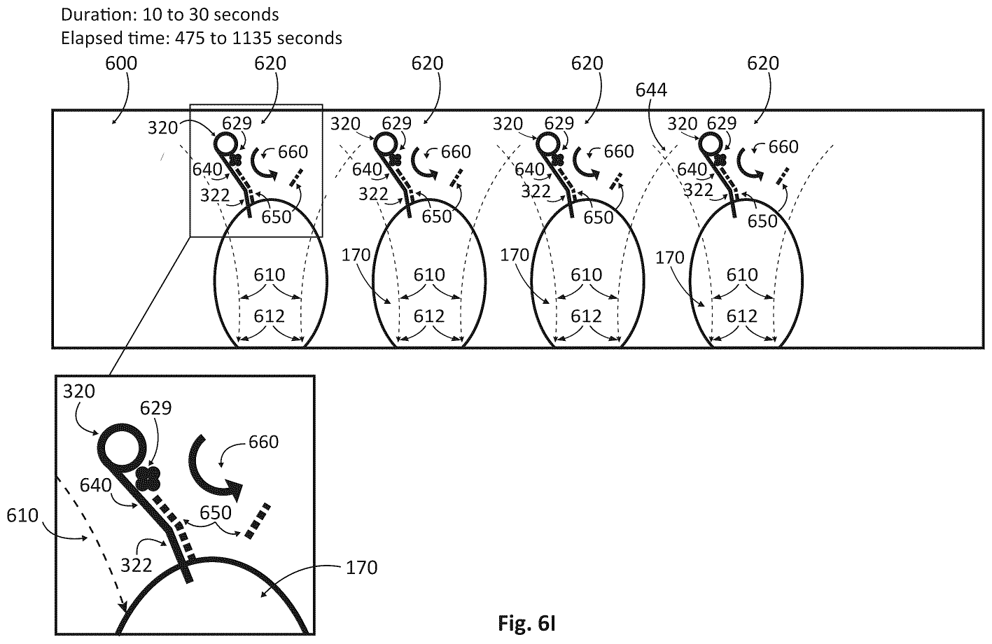

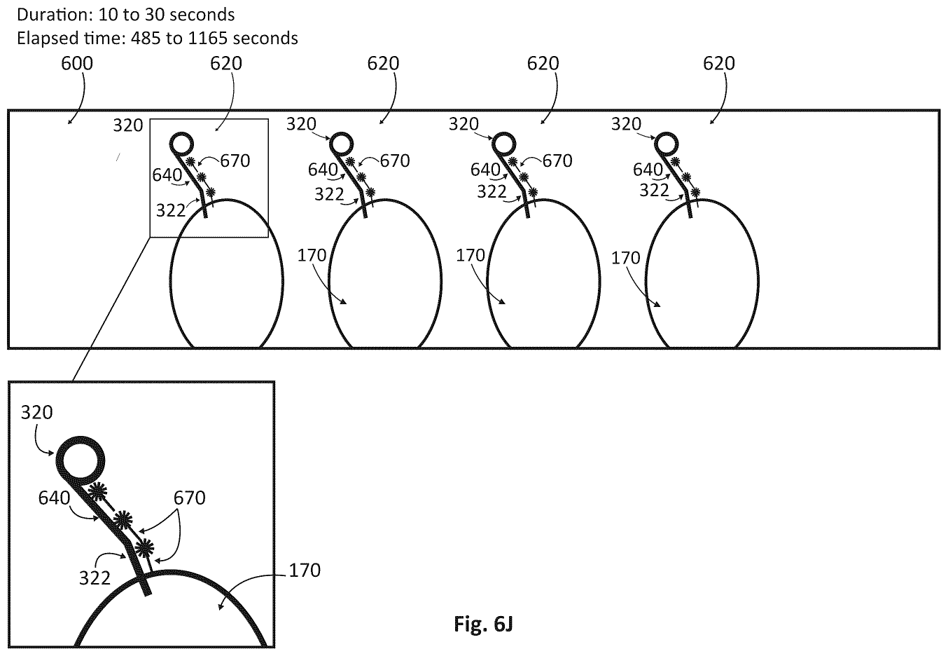

In a further configuration, the pre-anchored component is itself a pre-assembled complex, comprising a linear RCA probe pre-hybridized to a primer. In this scenario, the primer acts as an anchor, tethering its specific linear RCA probe partner to the hydrogel matrix. Using the “librarian” analogy, imagine the librarian holding not just a keyword, but a pre-assembled search tool containing a unique sentence from the book, ready for an instant match. This is shown generally in FIGS. 6A-6J, where forward primers (322) pre-hybridized to RCA probes (320, understood as linear before target-dependent ligation) are on the microgel (190 in FIG. 6A). Alternatively, as covered by embodiments, target-specific linear RCA probes themselves can be pre-anchored.

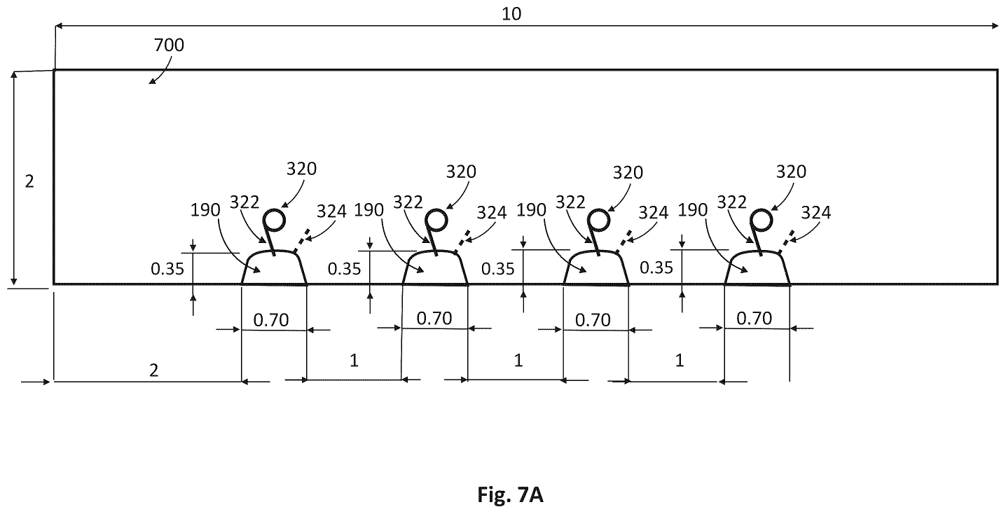

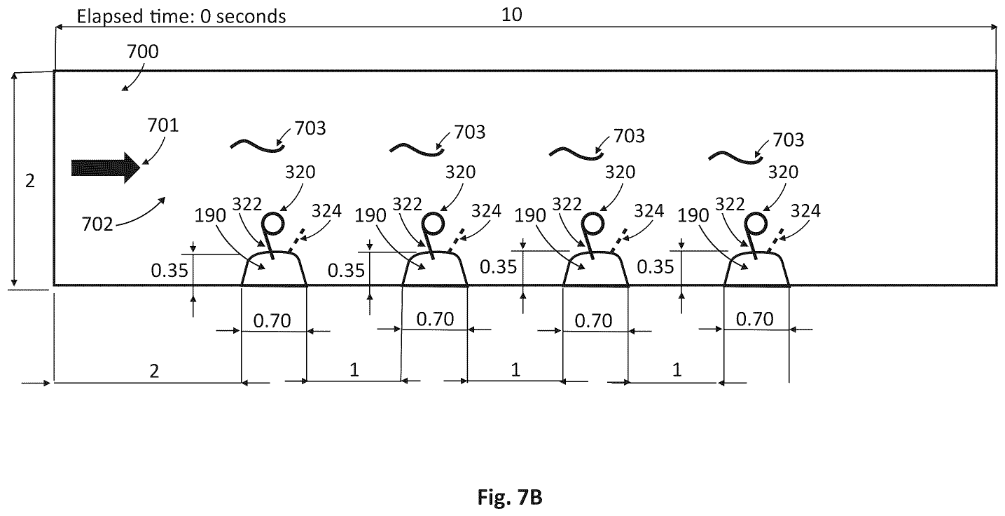

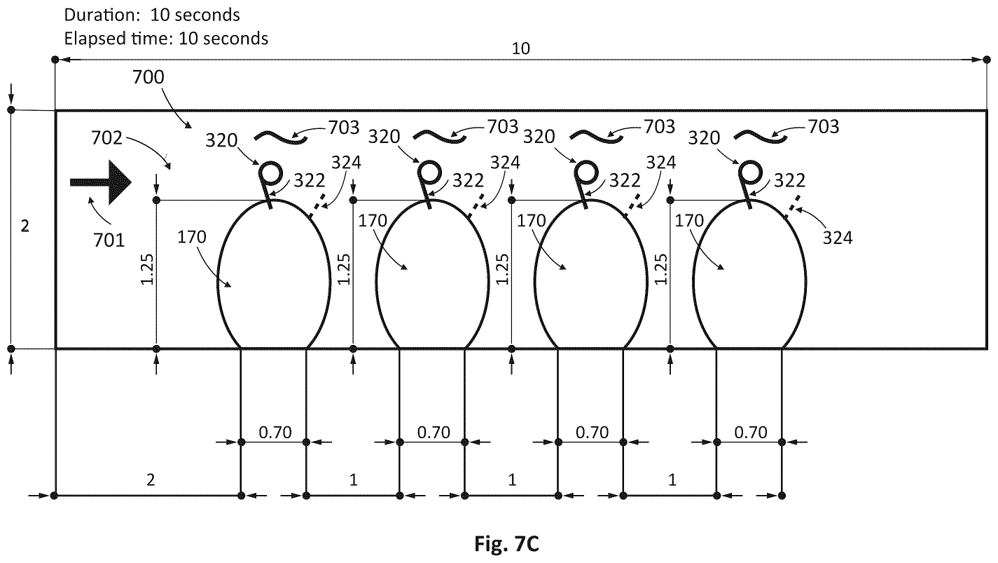

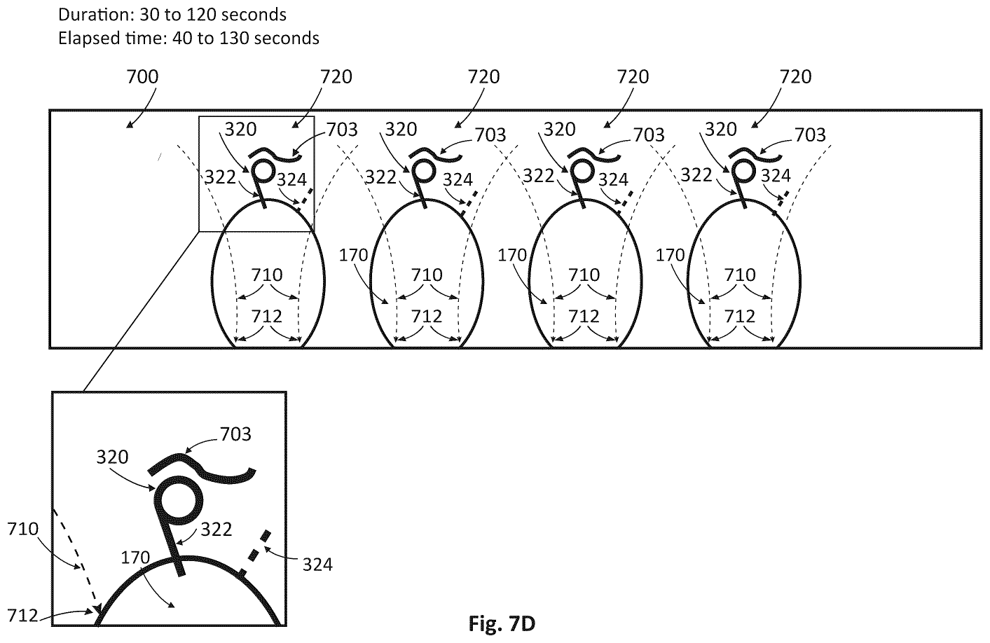

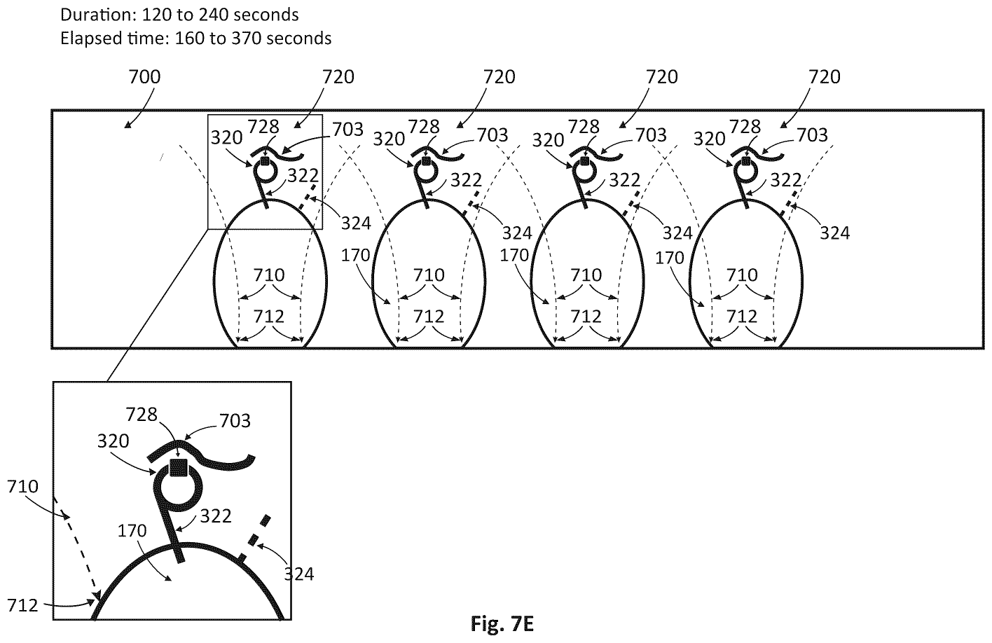

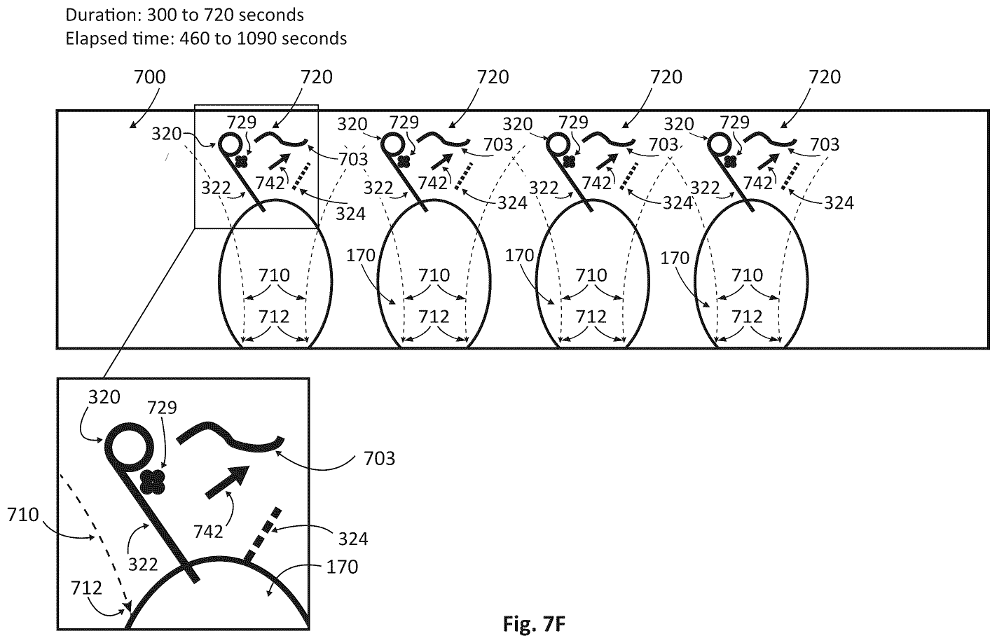

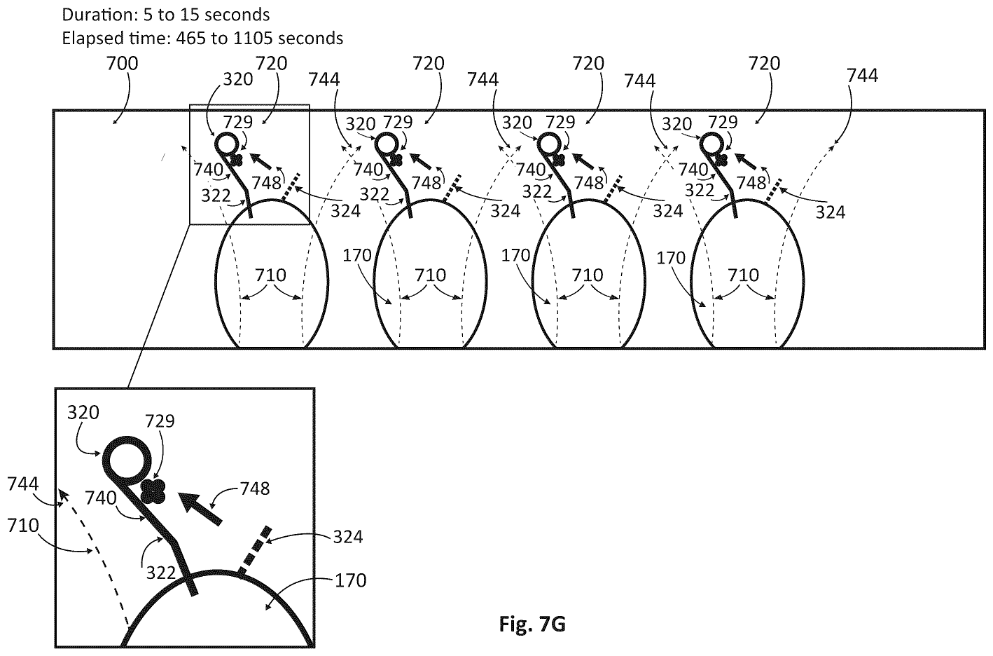

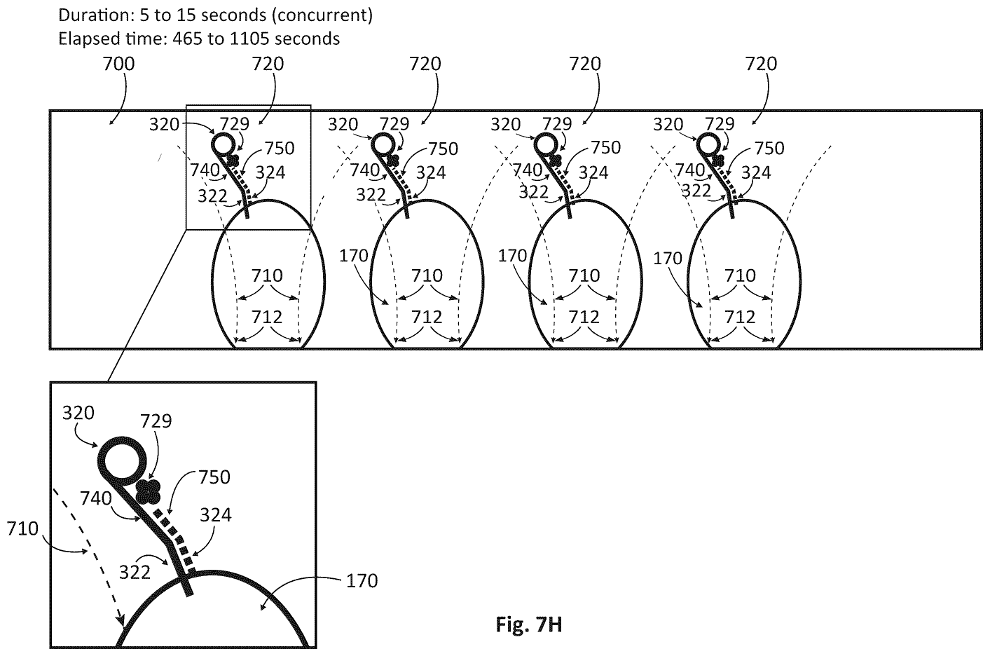

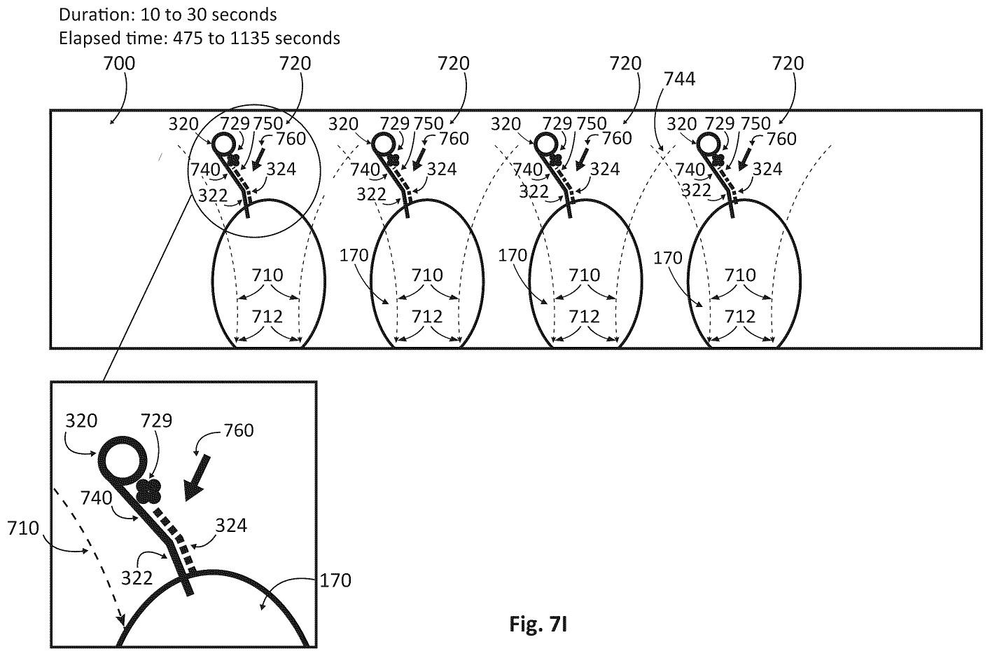

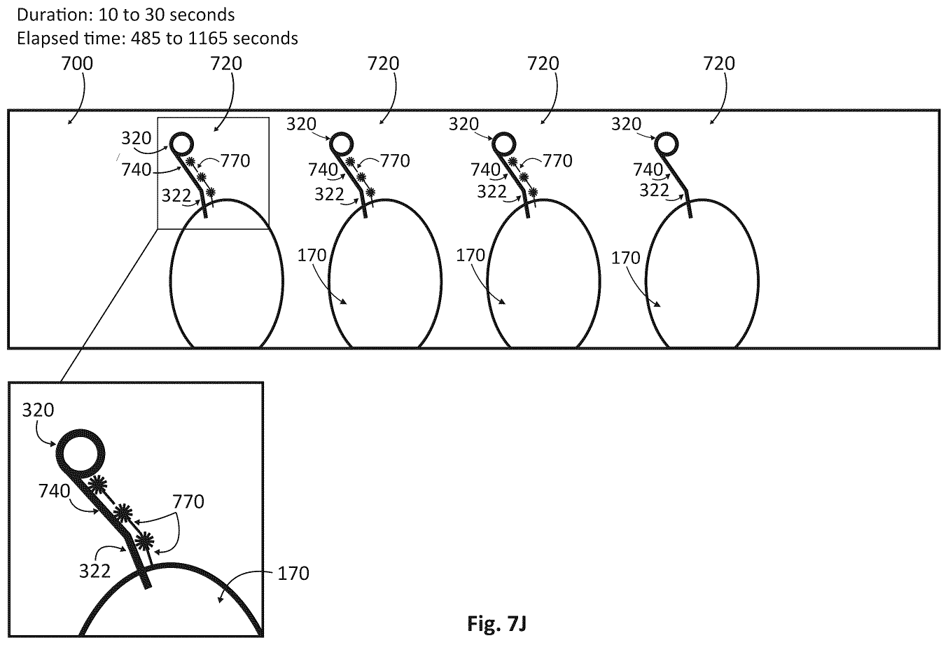

In yet another advanced setup, both forward primers and reverse primers for the RCA reaction can be pre-anchored within the microgel. This corresponds to the detailed method described in FIGS. 7A-7J and used in all the working Examples 1-5, where forward primers (322), RCA probes (320, functionally linear prior to ligation), and reverse primers (324) are all pre-anchored to the microgel deposits (190 in FIG. 7A).

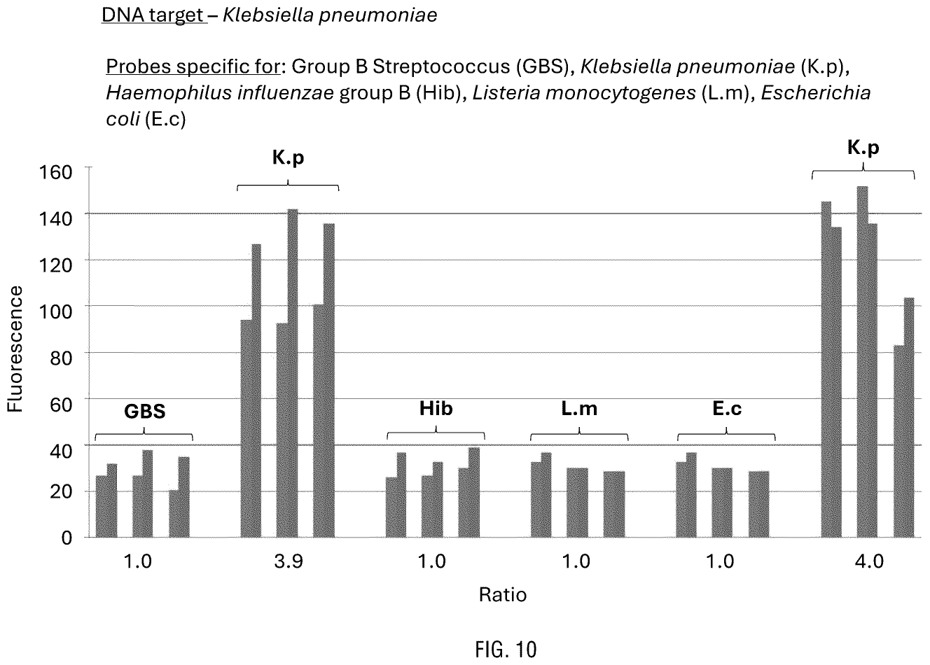

This ability to pre-load different specific search tools at different microgel locations on the same array is what enables multiplex detection—the “library” can simultaneously search for many different “books”. Examples 3, 4, and 5, which are illustrated in FIGS. 10, 11, and 12, respectively, directly demonstrate this capability by detecting Klebsiella pneumoniae and Listeria monocytogenes, including their simultaneous detection.

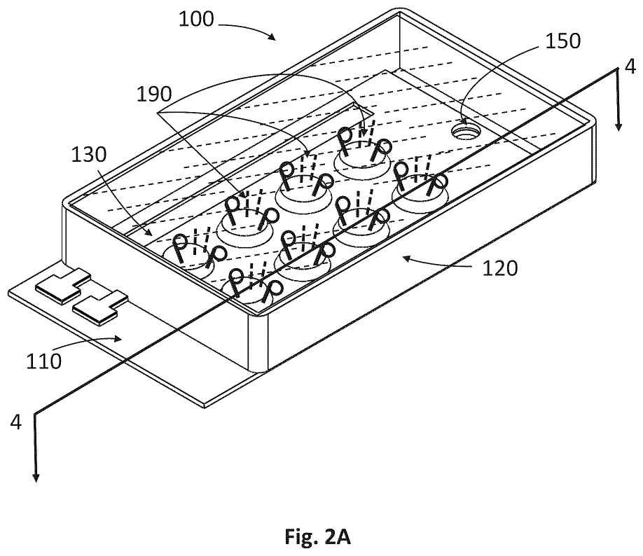

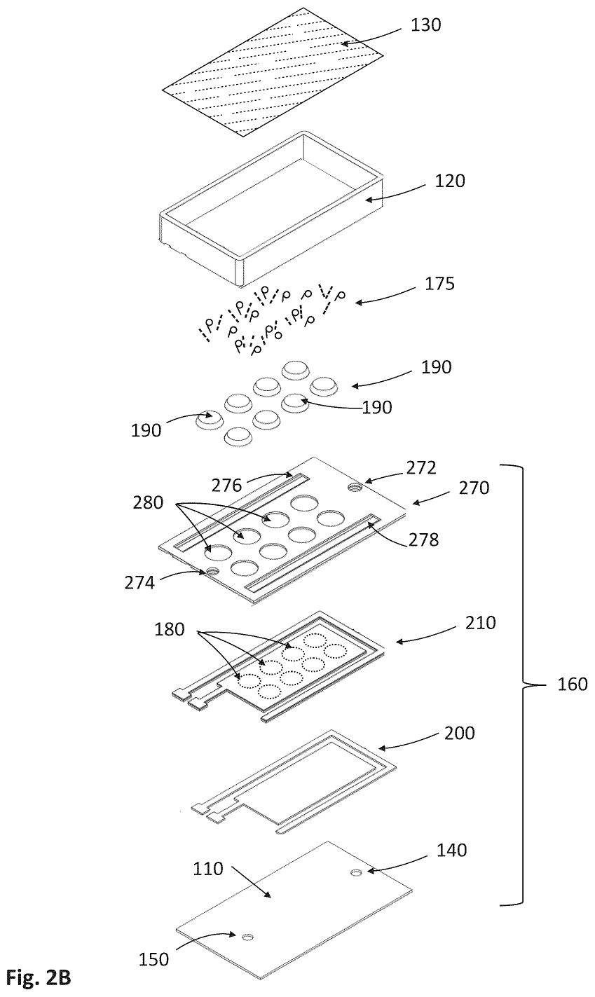

These microgel “search stations” can be prepared and stored in a dehydrated state (190 in FIGS. 2A, 2B, 4A, 5A, 6A, and 7A), like librarians at their posts, ready and waiting. This often allows for room-temperature shelf-storability of the electrophoretic array. Upon introduction of the sample solution (402, 502, 602, 702 in FIGS. 4B, 5B, 6B, and 7B), these microgels rapidly rehydrate (in about 10 seconds, as described in the parent specification), swelling to assume their functional, often generally hemispherical shape (170 in FIGS. 1A, 1B, 4C, 5C, 6C, and 7C) and activating their “search” capabilities.

A significant advantage of the invention is the use of electric fields to overcome the slow process of diffusion. Instead of passively waiting for target nucleic acids (the “books”) to randomly drift to the correct microgel station, the system acts like an “express delivery service” or a powerful announcement system within the library. As depicted generally (field lines 410 in FIG. 4D), when a voltage is applied across the electrodes of the array, the negatively charged nucleic acids in the sample solution are actively and rapidly driven towards and into the porous structure (interconnected void spaces) of the microgel deposits. This dramatically reduces the time needed for the target to find its specific “librarian”.

These electric fields serve multiple purposes throughout the process:

### 1) Target Delivery and Hybridization.

Driving all nucleic acid molecules, including target and non-target molecules from the bulk solution into the microgel regions (as illustrated conceptually in FIGS. 4D, 5D, 6D, and 7D) to encounter the pre-anchored components.

### 2) Concentration of Intermediates.

Driving hybridisation products (e.g., target molecule-RCA probe complexes) towards anchored molecules within the microgel, wherein the electric field strength is selected to be sufficient for electrophoretic transport while being below the threshold that would cause significant denaturation of the hybridized complex.

### 3) Enhancing Reaction Kinetics.

The localized concentration and manipulation by electric fields can enhance the speed of enzymatic activities within the porous structure of the microgel. This is achieved primarily by increasing the local concentration of reactants by actively transporting them into the confined volume of the microgel. The use of electric fields to accelerate biological reactions on microarrays is a known principle, described, for example, in U.S. Pat. No. 6,238,624.

### 4) Amplicon Concentration and Retention.

During and after amplification, electric fields are used to concentrate generated amplicons within said hydrogel microgel deposit. This can involve compressing the amplicons (e.g., 440 and 450 in FIGS. 4G and 4H leading to concentration in FIGS. 4I; 540 and 550 in FIGS. 5G and 5H to FIG. 5I; 640 and 650 in FIGS. 6G and 6H to FIG. 6I; and 740 and 750 in FIGS. 7G and 7H to FIG. 7I) against the microgel or an electrode surface, keeping the “photocopies” neatly stacked and preventing them from diffusing away, which is crucial for sensitive detection. This is a key application of electric fields during the RCA step itself. The general principle of using electric fields to concentrate charged biomolecules at specific locations on an electronic microarray is also described, for example, in U.S. Pat. No. 6,238,624.

### 5) Stringency or Cleaning.

Optionally, reversing polarity or applying specific field patterns can help remove non-specifically bound molecules, improving the cleanliness of the “search”. A specific embodiment of this step is described in Example 2 herein, where reverse polarity is used for removing non-specifically bound DNA. The general principle of using electric fields to assist in washing or purification steps on microelectronic arrays is also described, for example, in U.S. Pat. No. 6,238,624.

Once the correct target nucleic acid (the right “book”) arrives at its specifically matched microgel station (the “librarian” with the right pre-anchored tools), a cascade of rapid biochemical reactions takes place, predominantly within the localized reaction environment provided by the hydrogel microgel deposit (microgel hub), which is similar to a rapid photocopying process. The target nucleic acid (403 in FIGS. 4D and 703 in FIG. 7D) specifically binds or hybridizes to the pre-anchored components or to solution-phase components that are then captured. This forms a ligation-competent complex involving the target, a linear RCA probe (e.g., 320, which although depicted sometimes as circular in overview in the parent application's figures, is functionally linear before its target-dependent ligation), and at least one primer (e.g., 322). This complex formation is a highly specific recognition event, ensuring that only the correct “book” proceeds to the next step.

The next step is ligation, where the “padlock” closes, and this is a critical step. The linear RCA probe, now precisely aligned on its target, has its 5′ and 3′ ends brought into close proximity. A ligation enzyme, e.g., T4 ligase (428 in FIGS. 4E and 728 in FIG. 7E), is introduced (or is already present) and seals the gap, forming a covalently closed circular RCA probe template. This circularization is typically target-dependent, acting like a padlock that only closes if the correct key (the target sequence) is present. This step occurs before the main amplification, ensuring high fidelity.

Following ligation, a polymerase enzyme, e.g., Bst polymerase (429 in FIG. 4F or 729 in FIG. 7F), and nucleotides (dNTPs) are introduced or become active. The polymerase recognizes a primer (which might be pre-anchored or part of the initial complex) hybridized to the newly formed circular RCA probe template. It then begins to “roll” around this circle continuously synthesizing a long, single-stranded DNA molecule that is a tandem repeat of the circular template's complementary sequence. This results in a large amplicon (440 in FIG. 4F and 740 in FIG. 7F) that often remains associated with or tethered to the microgel region, especially with the help of applied electric fields.

If reverse primers (324) are also present, either pre-anchored in the microgel or introduced with the polymerase, they can bind to the primary amplicon and initiate further rounds of synthesis, leading to an even more rapid, potentially exponential, accumulation of amplicons (e.g., additional amplicons 450 in FIG. 4H and 750 in FIG. 7H). The entire amplification process occurs rapidly within the optimized porous structure of the hydrogel microgel deposit.

After a very short amplification period, all within the overall sub-20-minute timeframe (and in some embodiments, 8-15 minutes), the accumulated amplicons, concentrated within their respective microgel “search stations,” are detected. While the examples herein demonstrate completion in approximately 16-17 minutes, it is contemplated that this time can be reduced to 15 minutes or less. This reduction can be achieved under various conditions or through active optimization of the amplification phase.

For instance, the time-to-detection is inherently dependent on the initial concentration of the target nucleic acid in the sample. Samples with a higher target concentration will result in a higher concentration of ligated circular probes serving as templates for amplification. This larger number of initial templates leads to a faster accumulation of amplicons, allowing the detection threshold to be reached in a shorter time.

Furthermore, the amplification phase can be actively accelerated to reduce the overall assay time, for example, by reducing the duration of the polymerase step (as shown in FIG. 7F) from approximately 720 seconds to 600 seconds or less. Such a reduction in time can be facilitated by one or more of the following approaches:

### 1) Increasing Reverse Primer Concentration

In embodiments employing exponential RCA (such as that of FIG. 7), adding a higher concentration of reverse primers provides more initiation sites on the primary rolling circle amplicons, thereby increasing the rate of secondary strand synthesis and accelerating the overall accumulation of detectable products.

2) Modifying Enzyme and Substrate Concentrations

The reaction can be accelerated by slightly increasing the concentration of the polymerase enzyme and/or the dNTPs in the polymerase solution.

### 3) Employing a More Processive Polymerase

The use of an alternative DNA polymerase with higher processivity or faster strand-displacement activity (e.g., Phi29 polymerase or engineered variants) can significantly shorten the time required to generate long amplicons.

### 4) Optimizing the Reaction Buffer

The reaction buffer composition can be further optimized by including amplification enhancers known in the art to improve the efficiency of isothermal reactions. Such compounds can include, but are not limited to, dimethyl sulfoxide (DMSO), polyethylene glycol (PEG), or betaine, which are known to improve polymerase processivity and resolve inhibitory secondary structures in nucleic acid templates or amplicons.

By employing one or more of these strategies, the method can be reliably performed within the approximately 8 to 15-minute timeframe recited in present embodiments.

Typically, reporter molecules are introduced, for example, fluorescently labelled oligonucleotides (470 in FIG. 4J and 770 in FIG. 7J) that are complementary to the repeating sequence in the RCA amplicons). These reporters bind to the amplicons. If a target was present at a specific microgel station, that station will now contain a high concentration of amplicons and thus bound reporters, generating a strong, localized signal—the “found it!” signal.

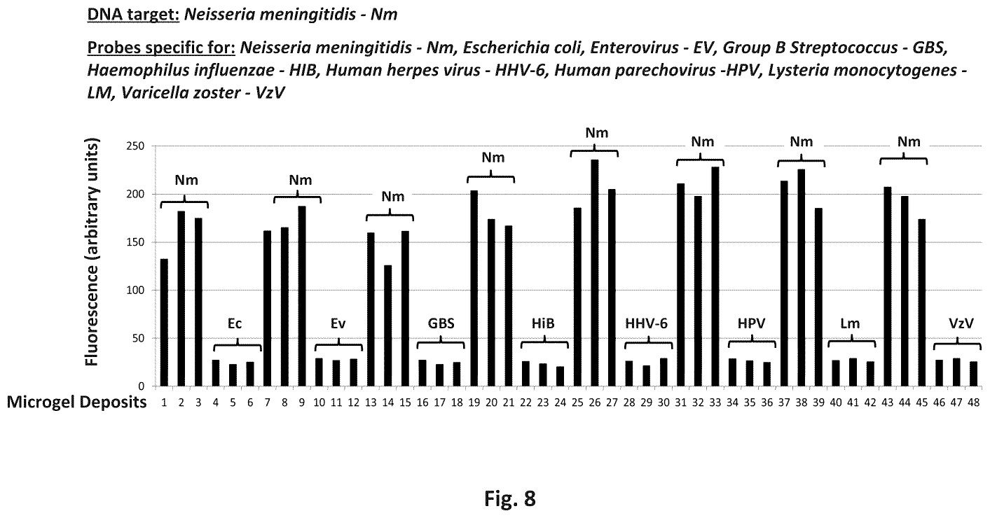

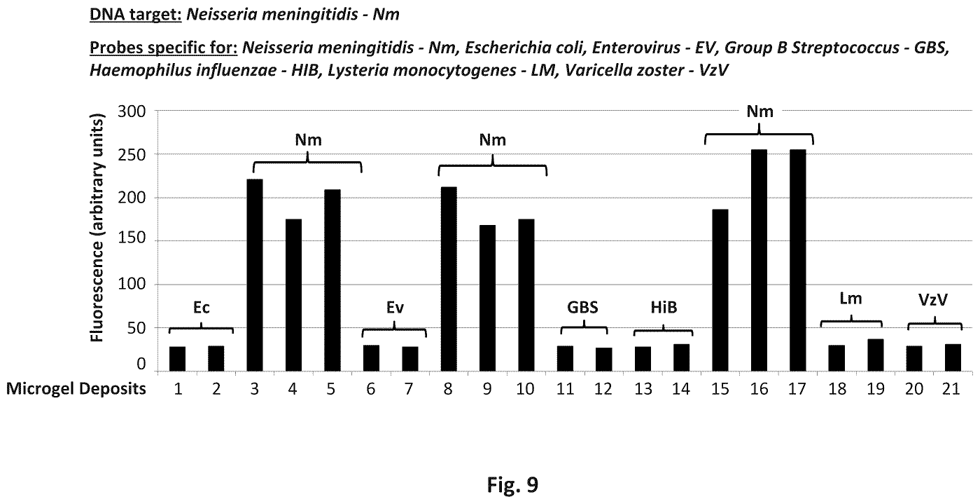

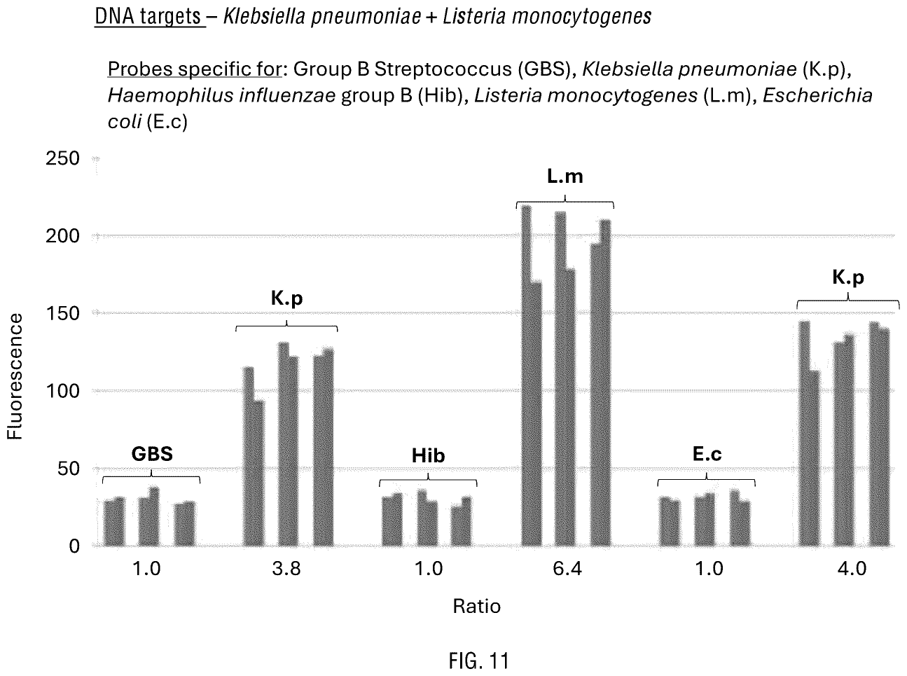

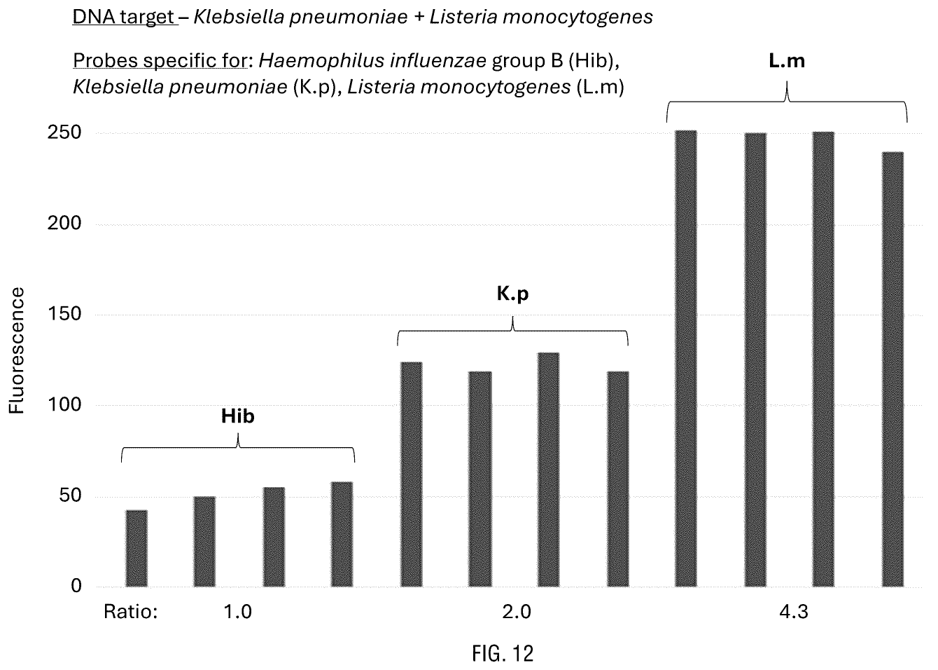

This signal, often fluorescence, is then read by a detection system. Because each microgel region is spatially distinct and pre-programmed for a specific target, observing a signal at a particular location on the array indicates the presence of that specific target in the original sample. The results from the examples, which are presented in FIGS. 8-12, unambiguously demonstrate this principle, where specific microgel spots light up corresponding to DNA targets introduced: for example, Neisseria meningitidis shown in FIGS. 8 and 9; Klebsiella pneumoniae in FIG. 10; and co-detection of Klebsiella pneumoniae and Listeria monocytogenes in FIGS. 11 and 12.

The central advancement emphasized in the present invention lies in the defined three-dimensional porous hydrogel matrix structure of the microgels and how this structure, particularly when formed as a porogen-derived pore network, is optimized for rapid molecular transport and for providing a localized reaction environment. This is a significant step beyond just using a generic gel spot. The optimized porous structure transforms the microgel into a highly efficient, three-dimensional true reaction hub. It's not just a surface; reactions occur throughout its volume. This localization and the high effective surface area within the pores concentrate reactants (targets, probes, primers, enzymes, dNTPs), dramatically speeding up binding and enzymatic reaction rates compared to bulk solution or simple 2D surfaces.

The defined porosity, resulting for instance from the use of a porogen during in-situ polymerization, is engineered to allow enhanced transport control, i.e., swift entry of necessary molecules (including nucleic acid targets up to at least 800 base pairs in length) driven by diffusion or, more importantly, by the applied electric fields, while also aiding in the retention and concentration of the large RCA amplicons. This prevents loss of signal and ensures that the “photocopies” stay at their designated “search station”.

The structured microgel provides a stable, yet permeable, interface with the electrodes (e.g., carbon electrodes). This allows electric fields to effectively permeate the microgel, driving molecules directly to and within the reaction sites and manipulating products efficiently.

The robust, anchored nature of the microgels, facilitated by the inclusion of an affinity-binding molecule like modified streptavidin, allows for reliable pre-loading of different specific biotinylated oligonucleotide components in different regions, facilitating robust multiplexed assays, as now supported by the new working examples (Examples 3-5, FIGS. 10-12). The potential for dehydration and room-temperature storage further enhances their practical utility.

In essence, the present invention, particularly through the innovations embodied herein, leverages the synergistic combination of:
(a) specifically structured and optimized hydrogel microgel reaction hubs formed, for example, from polymerizable solutions containing porogens and affinity-binding molecules;
(b) the precise spatial definition and multiplexing capability of electrophoretic arrays;
(c) the rapid and targeted molecular transport driven by electric fields; and
(d) the high amplification yield of RCA coupled with target-dependent ligation.

This combination allows for the detection of a plurality of specific nucleic acid sequences from samples with unprecedented speed (often under 20 minutes) and sensitivity. The microgels are no longer just passive anchors but are engineered as dynamic, “intelligent search stations” that significantly accelerate and localize the entire detection process, making this technology highly suitable for demanding diagnostic applications. The examples for diverse pathogens further underscore the broad applicability and robustness of this advanced approach.

### EXAMPLES

The following examples are illustrative of the practice of the invention and are not intended to be limiting.

### Example 1

### Detection of Meningitis Pathogens Employing the Method of FIGS. 7A-7J and Using a Synthetic DNA Target Molecule Representing Neisseria meningitidis

An electrophoretic array assembly similar to electrophoretic array assembly (700) (see FIG. 7A) containing 100 immobilized dried microgel deposits (190) having a base diameter of 0.45 mm and a height of approximately 0.2-0.3 mm was provided. 48 immobilized dried microgel deposits (190) were spotted with DNA target-specific RCA circular probes (320) pre-hybridized to forward primers (322) each specific for a different pathogen RCA probe and with reverse primers (324) each relevant to detection of a different pathogen, including inter alia Neisseria meningitidis.

Accordingly, the 48 spotted immobilized dried target molecule-specific microgel deposits (190) were target molecule-specific as follows:
Deposit 1—Specific to Neisseria meningitidis
Deposit 2—Specific to Neisseria meningitidis
Deposit 3—Specific to Neisseria meningitidis
Deposit 4—Specific to Escherichia coli
Deposit 5—Specific to Escherichia coli
Deposit 6—Specific to Escherichia coli
Deposit 7—Specific to Neisseria meningitidis
Deposit 8—Specific to Neisseria meningitidis
Deposit 9—Specific to Neisseria meningitidis
Deposit 10—Specific to Enterovirus
Deposit 11—Specific to Enterovirus
Deposit 12—Specific to Neisseria meningitidis
Deposit 13—Specific to Neisseria meningitidis
Deposit 14—Specific to Neisseria meningitidis
Deposit 15—Group B Streptococcus
Deposit 16—Group B Streptococcus
Deposit 17—Group B Streptococcus
Deposit 18—Specific to Neisseria meningitidis
Deposit 19—Specific to Neisseria meningitidis
Deposit 20—Specific to Neisseria meningitidis
Deposit 21—Specific to Haemophilus influenzae
Deposit 22—Specific to Haemophilus influenzae
Deposit 23—Specific to Haemophilus influenzae
Deposit 24—Specific to Neisseria meningitidis
Deposit 25—Specific to Neisseria meningitidis
Deposit 26—Specific to Neisseria meningitidis
Deposit 27—Specific to Human herpes virus
Deposit 28—Specific to Human herpes virus
Deposit 29—Specific to Human herpes virus
Deposit 30—Specific to Human herpes virus
Deposit 31—Specific to Neisseria meningitidis
Deposit 32—Specific to Neisseria meningitidis
Deposit 33—Specific to Neisseria meningitidis
Deposit 34—Specific to Human parechovirus
Deposit 35—Specific to Human parechovirus
Deposit 36—Specific to Human parechovirus
Deposit 37—Specific to Neisseria meningitidis
Deposit 38—Specific to Neisseria meningitidis
Deposit 39—Specific to Neisseria meningitidis
Deposit 40—Specific to Lysteria monocytogenes
Deposit 41—Specific to Lysteria monocytogenes
Deposit 42—Specific to Lysteria monocytogenes
Deposit 43—Specific to Neisseria meningitidis
Deposit 44—Specific to Neisseria meningitidis
Deposit 45—Specific to Neisseria meningitidis
Deposit 46—Specific to Varicella zoster
Deposit 47—Specific to Varicella zoster

A solution (702) containing nucleic acid target molecules (703) (with concentration of 100 nM) representing Neisseria meningitidis was supplied to the interior volume of the electrophoretic array, at a time defined as T₀. The solution (702) also included a low conductivity buffer supporting rapid DNA transport and hybridization to the RCA probes deposited on the microgels.

Supplying solution (702) caused dried target molecule-specific microgel deposits (190) to assume their hydrated state, designated by reference numeral (170), after a duration of 10 seconds (FIGS. 7B and 7C).

At time T=T₀+10 seconds, a constant current of 1.6 mA was applied across the working and counter electrode contacts (260) and (250) respectively, resulting in voltages of 4.5 V, yielding an electric field applied across the electrophoretic array of 12.5 V per cm and producing electrophoretic addressing (FIG. 7D). The duration of the electro-phoretic addressing was 40 seconds.

At time T=T₀+50 seconds, a ligation reaction solution including ligation reaction enzyme T4 ligase (Blunt T/A, from New England Biolabs) was supplied to the interior volume of the electrophoretic array, replacing solution (702), for a duration of approximately 180 seconds (FIG. 7E).

At time T=T₀+230 sec., a polymerase solution containing Bst polymerase enzyme (729) and dNTPs (from New England Biolabs) was supplied to the interior volume of the electrophoretic array, replacing the ligation reaction solution, for a duration of approximately 720 seconds (FIG. 7F).

At time T=T₀+950 sec., a constant current of 1.6 mA was applied across the working and counter electrode contacts (260) and (250) respectively, resulting in voltages of 4.5 V, yielding an electric field applied across the electrophoretic array of 12.5 V per cm and providing recapture of RCA amplicons from the polymerase solution. The duration of this step was approximately 20 seconds (FIG. 7I).

At time T=T₀+970 sec., a red reporter solution containing fluorescently labelled oligonucleotides (Alexa 647 from Integrated Device Technology, Inc., San Jose, Calif.) was supplied to the interior volume of the electrophoretic array, replacing the polymerase solution for a duration of approximately 30 seconds (FIG. 7J).

Following washing out of the red reporter solution, a fluorescence image of the electrophoretic array assembly (700) was taken via window (130) and the presence of nucleic acid target molecules (703) representing Neisseria meningitidis was detected at the following ones of immobilized dried target molecule-specific microgel deposits (170): 1-3, 7-9, 13-15, 19-21, 25-27, 31-33, 37-39, and 43, 44-45. The presence of nucleic acid target molecules (703) representing Neisseria meningitidis was not detected at the following ones of immobilized dried target molecule-specific microgel deposits (170): 4-6, 10-12, 16-18, 22-24, 28-30, 34-36, 40-42, and 46-48.

The detection results are summarized in FIG. 8. It is noted that the average ratio of intensities of the fluorescence signal obtained from deposits 1-3, 7-9, 13-15, 19-21, 25-27, 31-33, 37-39, and 43, 44-45 to the fluorescence signal obtained from deposits 4-6, 10-12, 16-18, 22-24, 28-30, 34-36, 40-42, and 46-48 was approximately 8.5.

### Example 2

### Detection of Meningitis Pathogens Employing the Method of FIGS. 7A-7J and Using a Genomic DNA Target Molecule Extracted From Neisseria meningitidis Pathogen Spiked Into Cerebrospinal Fluid Clinical Sample

An electrophoretic array assembly similar to electrophoretic array assembly (700) (FIG. 7A) containing 100 immobilized dried microgel deposits (190) having a base diameter of 0.45 mm and a height of approximately 0.2-0.3 mm was provided. 21 immobilized dried microgel deposits (190) were spotted with RCA circular probes (320) pre-hybridized to forward primers (322) each specific for a different pathogen RCA probe, including inter alia Neisseria meningitidis and with reverse primers (324) each relevant to detection of a different pathogen, including inter alia Neisseria meningitidis.

Accordingly, the 21 spotted immobilized dried target molecule-specific microgel deposits (190) were target molecule-specific as follows:
Deposit 1—Specific to Escherichia coli
Deposit 2—Specific to Escherichia coli
Deposit 3—Specific to Neisseria meningitidis
Deposit 4—Specific to Neisseria meningitidis
Deposit 5—Specific to Neisseria meningitidis
Deposit 6—Specific to Enterovirus
Deposit 7—Specific to Enterovirus
Deposit 8—Specific to Neisseria meningitidis
Deposit 9—Specific to Neisseria meningitidis
Deposit 10—Specific to Neisseria meningitidis
Deposit 11—Group B Streptococcus
Deposit 12—Group B Streptococcus
Deposit 13—Specific to Haemophilus influenzae
Deposit 14—Specific to Haemophilus influenzae
Deposit 15—Specific to Neisseria meningitidis
Deposit 16—Specific to Neisseria meningitidis
Deposit 17—Specific to Neisseria meningitidis
Deposit 18—Specific to Lysteria monocytogenes
Deposit 19—Specific to Lysteria monocytogenes
Deposit 20—Specific to Varicella zoster
Deposit 21—Specific to Varicella zoster

A clinical sample of cerebrospinal fluid (CSF) was spiked with Neisseria meningitides pathogen, and genomic DNA extraction performed using a common magnetic bead-based DNA extraction method. The input concentration of DNA target in cerebrospinal fluid was determined by a reference real-time PCR method that yielded Neisseria meningitides pathogen concentration in clinical sample of cerebrospinal fluid of 720 copies of DNA per microliter of CSF as input.

A solution (702), prepared from the spiked clinical sample, was supplied to the interior volume of the electrophoretic array, at a time defined as T₀. The solution (702) also included a low conductivity buffer supporting rapid DNA transport and hybridization to the RCA probes deposited on the microgels.

Supplying solution (702) caused dried target molecule-specific microgel deposits (190) to assume their hydrated state, designated by reference numeral (170), after a duration of 10 seconds (FIGS. 7B-7C).

At time T=T₀+10 sec., a constant current of 1.6 mA was applied across the working and counter electrode contacts (260) and (250) respectively, resulting in voltages of 4.5 V, yielding an electric field applied across the electrophoretic array of 12.5 V per cm and producing electrophoretic addressing (FIG. 7D). The duration of the electrophoretic addressing was 40 seconds.

At time T=T₀+50 sec., a reverse polarity electric field was applied by applying a constant current of negative 1.6 mA across the working and counter electrode contacts (260) and (250) respectively, resulting in voltages of 4.5 V, yielding an electric field applied across the electrophoretic array of 12.5 V per cm and enhancing removal of non-specifically bound DNA targets. The duration of the electrophoretic addressing was 10 seconds (FIG. 7G).

At time T=T₀+60 sec., a ligation reaction solution including ligation reaction enzyme T4 ligase (Blunt T/A, from New England Biolabs) was supplied to the interior volume of the electrophoretic array, replacing solution (702), for a duration of approximately 180 seconds (FIG. 7E).

At time T=T₀+240 sec., a polymerase solution containing Bst polymerase enzyme (729) and dNTPs (from New England Biolabs) was supplied to the interior volume of the electrophoretic array, replacing the ligation reaction solution, for a duration of approximately 720 seconds (FIG. 7F).

At time T=T₀+960 sec., a constant current of 1.6 mA was applied across the working and counter electrode contacts (260) and (250) respectively, resulting in voltages of 4.5 V, yielding an electric field applied across the electrophoretic array of 12.5 V per cm and providing recapture of RCA amplicons from the polymerase solution. The duration of this step was approximately 20 seconds (FIG. 7I).

At time T=T₀+980 sec., a red reporter solution containing fluorescently labelled oligonucleotides (Alexa 647 from Integrated Device Technology, Inc., San Jose, Calif.) was supplied to the interior volume of the electrophoretic array, replacing the polymerase solution for a duration of approximately 30 seconds (FIG. 7J).

Following washing out of the red reporter solution, a fluorescence image of the electrophoretic array assembly (700) was taken via window (130) and the presence of nucleic acid target molecules (703) representing Neisseria meningitidis was detected at the following ones of immobilized dried target molecule-specific microgel deposits (170): 3-5, 8-10, and 15-17. The presence of nucleic acid target molecules (703) representing Neisseria meningitidis was not detected at the following ones of immobilized dried target molecule-specific microgel deposits (170): 1, 2, 6, 7, 11, 12, 13, 14, 18, 19, 20, and 21.

The detection results are summarized in FIG. 9. It is noted that the average ratio of intensities of the fluorescence signal obtained from deposits 3-5, 8-10, and 15-17 to the fluorescence signal obtained from deposits 1, 2, 6, 7, 11, 12, 13, 14, 18, 19, 20, and 21 was approximately 4.3.

### Example 3

### Detection of Klebsiella pneumoniae Pathogen With the Method of FIGS. 7A-7J and Using a Genomic DNA Target Molecule Extracted From Klebsiella Pneumoniae

An electrophoretic array assembly similar to electrophoretic array assembly (700) (FIG. 7A) containing 100 immobilized dried microgel deposits (190) having a base diameter of 0.45 mm and a height of approximately 0.2-0.3 mm was provided. 36 immobilized dried microgel deposits (190) were spotted with DNA target-specific RCA circular probes (320) pre-hybridized to forward primers (322) each specific for a different pathogen RCA probe, including inter alia Klebsiella pneumoniae and with reverse primers (324) each relevant to detection of a different pathogen, including inter alia Klebsiella pneumoniae.

Accordingly, the 36 spotted immobilized dried target molecule-specific microgel deposits (190) were target molecule-specific as follows:
Deposit 1—Specific to Group B Streptococcus
Deposit 2—Specific to Group B Streptococcus
Deposit 3—Specific to Group B Streptococcus
Deposit 4—Specific to Group B Streptococcus
Deposit 5—Specific to Group B Streptococcus
Deposit 6—Specific to Group B Streptococcus
Deposit 7—Specific to Klebsiella pneumoniae
Deposit 8—Specific to Klebsiella pneumoniae
Deposit 9—Specific to Klebsiella pneumoniae
Deposit 10—Specific to Klebsiella pneumoniae
Deposit 11—Specific to Klebsiella pneumoniae
Deposit 12—Specific to Klebsiella pneumoniae
Deposit 13—Specific to Haemophilus influenzae group B
Deposit 14—Specific to Haemophilus influenzae group B
Deposit 15—Specific to Haemophilus influenzae group B
Deposit 16—Specific to Haemophilus influenzae group B
Deposit 17—Specific to Haemophilus influenzae group B
Deposit 18—Specific to Haemophilus influenzae group B
Deposit 19—Specific to Listeria monocytogenes
Deposit 20—Specific to Listeria monocytogenes
Deposit 21—Specific to Listeria monocytogenes
Deposit 22—Specific to Listeria monocytogenes
Deposit 23—Specific to Listeria monocytogenes
Deposit 24—Specific to Listeria monocytogenes
Deposit 25—Specific to Escherichia coli
Deposit 26—Specific to Escherichia coli
Deposit 27—Specific to Escherichia coli
Deposit 28—Specific to Escherichia coli
Deposit 29—Specific to Escherichia coli
Deposit 30—Specific to Escherichia coli
Deposit 31—Specific to Klebsiella pneumoniae
Deposit 32—Specific to Klebsiella pneumoniae
Deposit 33—Specific to Klebsiella pneumoniae
Deposit 34—Specific to Klebsiella pneumoniae
Deposit 35—Specific to Klebsiella pneumoniae
Deposit 36—Specific to Klebsiella pneumoniae

A solution (702) containing nucleic acid target molecules (703) (with concentration of 100 nM) of Klebsiella pneumoniae was supplied to the interior volume of the electrophoretic array, at a time defined as T₀. The solution (702) also included a low conductivity buffer supporting rapid DNA transport and hybridization to the RCA probes deposited on the microgels. Supplying solution (702) caused dried target molecule-specific microgel deposits (190) to assume their hydrated state, designated by reference numeral (170), after a duration of 10 seconds (FIGS. 7B and 7C).

At time T=T₀+10 sec., a constant current of 1.6 mA was applied across the working and counter electrode contacts (260) and (250) respectively, resulting in voltages of 4.5 V, yielding an electric field applied across the electrophoretic array of 12.5 V per cm and producing electrophoretic addressing (FIG. 7D). The duration of the electrophoretic addressing was 40 seconds.

At time T=T₀+50 sec., a ligation reaction solution including ligation reaction enzyme T4 ligase (Blunt T/A, from New England Biolabs) was supplied to the interior volume of the electrophoretic array, replacing solution (702), for a duration of approximately 180 seconds (FIG. 7E).

At time T=T₀+230 sec., a polymerase solution containing Bst polymerase enzyme (729) and dNTPs (from New England Biolabs) was supplied to the interior volume of the electrophoretic array, replacing the ligation reaction solution, for a duration of approximately 720 seconds (FIG. 7F).

At time T=T₀+950 sec., a constant current of 1.6 mA was applied across the working and counter electrode contacts (260) and (250) respectively, resulting in voltages of 4.5 V, yielding an electric field applied across the electrophoretic array of 12.5 V per cm and providing recapture of RCA amplicons from the polymerase solution. The duration of this step was approximately 20 seconds (FIG. 7I).

At time T=T₀+970 sec., a red reporter solution containing fluorescently labelled oligonucleotides (Alexa 647 from Integrated Device Technology, Inc., San Jose, CA) was supplied to the interior volume of the electrophoretic array, replacing the polymerase solution for a duration of approximately 30 seconds (FIG. 7J).

Following washing out of the red reporter solution, a fluorescence image of the electrophoretic array assembly (700) was taken via window (130) and the presence of nucleic acid target molecules (703) representing Klebsiella pneumoniae was detected at the following ones of immobilized dried target molecule-specific microgel deposits (170): 7-11, 12, and 31-36. The presence of nucleic acid target molecules (703) of Klebsiella pneumoniae was not detected at the following ones of immobilized dried target molecule-specific microgel deposits (170): 1-6 and 13-30.

The detection results are summarized in FIG. 10. It is noted that the average ratio of intensities of the fluorescence signal obtained from deposits 7-12 and 31-36 to the fluorescence signal obtained from deposits 1-6 and 13-30 was approximately 4.

### Example 4

### Simultaneous Detection of Klebsiella pneumoniae and Listeria monocytogenes Pathogens Employing the Method of FIGS. 7A-7J and Using Genomic DNA Targets Molecules Extracted From Klebsiella pneumoniae and Listeria monocytogenes

An electrophoretic array assembly similar to electrophoretic array assembly (700) (FIG. 7A) containing 100 immobilized dried microgel deposits (190) having a base diameter of 0.45 mm and a height of approximately 0.2-0.3 mm was provided. 36 immobilized dried microgel deposits (190) were spotted with DNA target-specific RCA circular probes (320) pre-hybridized to forward primers (322) each specific for a different pathogen RCA probe, including inter alia Klebsiella pneumoniae and Listeria monocytogenes and with reverse primers (324) each relevant to detection of a different pathogen, including inter alia Klebsiella pneumoniae and Listeria monocytogenes. Accordingly, the 36 spotted immobilized dried target molecule-specific microgel deposits (190) were target molecule-specific as follows (list as per Example 3).

A solution (702) containing nucleic acid target molecules (703) (100 nM concentration) of Klebsiella pneumoniae and Listeria monocytogenes was supplied to the interior volume of the electrophoretic array, at a time defined as T₀. The solution (702) also included a low conductivity buffer supporting rapid DNA transport and hybridization to the RCA probes deposited on the microgels. Supplying solution (702) caused dried target molecule-specific microgel deposits (190) to assume their hydrated state, designated by reference numeral (170), after a duration of 10 seconds (FIGS. 7B and 7C).

The subsequent steps of electrophoretic addressing, ligation, polymerization, recapture of amplicons, and reporter addition were performed as described in Example 3 (referencing FIGS. 7D, 7E, 7F, 7I and 7J respectively). Following washing out of the red reporter solution, a fluorescence image of the electrophoretic array assembly (700) was taken via window (130). The presence of nucleic acid target molecules (703) representing Klebsiella pneumoniae was detected at the following ones of immobilized dried target molecule-specific microgel deposits (170): 7-12 and 31-36, and simultaneously the presence of nucleic acid target molecules (703) representing Listeria monocytogenes was detected at the following ones of immobilized dried target molecule-specific microgel deposits (170): 19-24.

The presence of nucleic acid target molecules (703) of Klebsiella pneumoniae or Listeria monocytogenes was not detected at the following ones of immobilized dried target molecule-specific microgel deposits (170): 1-6, 13-18 and 25-30. The detection results are summarized in FIG. 11. It is noted that the average ratio of intensities of the fluorescence signal obtained from deposits 7-12 and 31-36 (specific for K. pneumoniae) to the fluorescence signal obtained from non-target control deposits (1-6, 13-18, and 25-30) was 3.8, while the average ratio of intensities of the fluorescence signal obtained from deposits 19-24 (specific for L. monocytogenes) to the fluorescence signal obtained from said non-target control deposits was 6.4.

### Example 5

### Simultaneous Detection of Klebsiella pneumoniae and Listeria monocytogenes Pathogens Employing the Method of FIGS. 5A-5J and Using Genomic DNA Targets Molecules Extracted From Klebsiella pneumoniae and Listeria monocytogenes

An electrophoretic array assembly similar to electrophoretic array assembly (500) (FIG. 5A) containing 100 immobilized dried microgel deposits (190) having a base diameter of 0.45 mm and a height of approximately 0.2-0.3 mm was provided. 12 immobilized dried microgel deposits (190) were spotted with forward primers (322) each specific for a different pathogen RCA probe, including inter alia Klebsiella pneumoniae and Listeria monocytogenes.

Accordingly, the 12 spotted immobilized dried target molecule-specific microgel deposits (190) were target molecule-specific as follows:
Deposit 1—Specific to Haemophilus influenzae group B
Deposit 2—Specific to Haemophilus influenzae group B
Deposit 3—Specific to Haemophilus influenzae group B
Deposit 4—Specific to Haemophilus influenzae group B
Deposit 5—Specific to Klebsiella pneumoniae
Deposit 6—Specific to Klebsiella pneumoniae
Deposit 7—Specific to Klebsiella pneumoniae
Deposit 8—Specific to Klebsiella pneumoniae
Deposit 9—Specific to Listeria monocytogenes
Deposit 10—Specific to Listeria monocytogenes
Deposit 11—Specific to Listeria monocytogenes
Deposit 12—Specific to Listeria monocytogenes

A solution (502) containing nucleic acid target molecules (503) (with concentration of 100 nM) of Klebsiella pneumoniae and Listeria monocytogenes was supplied to the interior volume of the electrophoretic array, at a time defined as T₀. This solution also included nucleic acid target molecule specific RCA circular probes (320) in addition to nucleic acid target molecules (503). The solution (502) also included a low conductivity buffer supporting rapid DNA transport and hybridization to the RCA probes deposited on the microgels. Supplying solution (502) caused dried target molecule-specific microgel deposits (190) to assume their hydrated state, designated by reference numeral (170), after a duration of 10 seconds (FIGS. 5B and 5C).

The subsequent steps of electrophoretic addressing (FIG. 5D), ligation (FIG. 5E), polymerization with reverse primer (324) supplied in the polymerase solution (FIG. 5F), recapture of amplicons (FIG. 5I), and reporter addition (FIG. 5J) were performed as generally described for the method of FIGS. 5A-5J, with specific timings and reagent details analogous to those in Example 3.

Following washing out of the red reporter solution, a fluorescence image of the electrophoretic array assembly (500) was taken via window (130). The presence of nucleic acid target molecules (503) representing Klebsiella pneumoniae was detected at the following ones of immobilized dried target molecule-specific microgel deposits (170): 5-8, and the presence of nucleic acid target molecules (503) representing Listeria monocytogenes was simultaneously detected at the following ones of immobilized dried target molecule-specific microgel deposits (170): 9-12.

The presence of nucleic acid target molecules of Klebsiella pneumoniae or Listeria monocytogenes was not detected at the following ones of immobilized dried target molecule-specific microgel deposits (170): 1-4 (specific for Haemophilus influenzae group B). A parallel similar experiment with Klebsiella pneumoniae or Listeria monocytogenes specific microgel deposits and no nucleic acid target molecules present in solution (502), yielded fluorescence signal intensity similar to the fluorescence signal intensity obtained from deposits 1-4, thereby designating signal intensities from deposits 1,2,3 and 4 as negative controls. The detection results are summarized in FIG. 12.

It is noted that the average ratio of intensities of the fluorescence signal obtained from deposits 5-8 (specific for K. pneumoniae) to the fluorescence signal obtained from deposits 1-4 (negative control) was approximately 2, while the average ratio of intensities of the fluorescence signal obtained from deposits 9-12 (specific for L. monocytogenes) to the fluorescence signal obtained from deposits 1-4 was 4.3.

### Example 6

### Protocol for Hydrogel Preparation and Spotting on Carbon Arrays

This example describes the procedures for preparing hydrogel and spotting it onto carbon arrays, as used in preferred embodiments of the invention for creating the structured microgel deposits. The protocol details the formation of the hydrogel microgel deposits containing acrylamide/BIS, the porogen APO 10, modified streptavidin for affinity binding, and cured using a photoinitiator and UV light, resulting in the structured microgels used in the described invention.

### Monomer Solution Preparation

1) Acrylamide (0.691g) and N,N′-Methylenebisacrylamide (BIS) (0.167g) were weighed and added to a 50 mL Falcon tube. (While weights may vary, the weight ratio should be maintained).
2) 2 mL of 5 mM sodium phosphate buffer (NaPO₄⁻), pH 7.0, containing 0.05% Proclin 300, was added to the tube. (The chemical to buffer volume ratio is maintained if chemical weights differ).
3) The acrylamide and BIS were dissolved by vigorous and repeated vortexing of the 50 mL tube.
4) The resulting acrylamide/BIS/NaPO₄⁻ solution was transferred into a 3 mL syringe fitted with a 0.2 μm syringe filter and filtered into a clean 15 mL conical tube.
5) 30 mg of APO 10 (which serves as a porogen in the hydrogel) was weighed into a brown 1.5 mL Eppendorf tube.
6) 225 μL of the filtered acrylamide/BIS solution (from step 4) was transferred to the brown tube containing the APO 10.
7) The brown tube (containing the mixture of acrylamide/BIS in NaPO₄⁻ and APO 10) was vortexed briefly. This solution constitutes the monomer solution.

### Photoinitiator Solution Preparation

1) A new 1.5 mL brown Eppendorf tube was pre-weighed.
2) Approximately 20 μL of Darocur 4265 stock solution was transferred to the pre-weighed tube using a P200 pipette tip whose pointed front half had been cut off to increase the tip opening (due to Darocur's high viscosity).
3) The brown tube was weighed again to determine the exact quantity of Darocur transferred.
4) DMSO was added to the brown tube containing Darocur 4265 to achieve an 8% (v/v) solution of Darocur in DMSO, assuming a density of 1.0 g/mL for Darocur (e.g., 460 μL of DMSO to 40 mg of Darocur).
5) The tube was vortexed well to create the Darocur solution for use in the hydrogel spotting solution.

### Hydrogel Spotting Solution Preparation

1) The monomer/APO10 solution, modified streptavidin solution, and the 8% Darocur in DMSO solution were combined in a 500 μL brown microcentrifuge tube according to the volumes specified (e.g., for 50 arrays: 66 μL monomer/APO10, 42 μL modified streptavidin, 12 μL 8% Darocur in DMSO, for a total of 120 μL, as per Table A of the protocol). (Volumes are scaled dependent on the number of arrays to be spotted).

TABLE A
Composition of hydrogel solution.

| | Volume | | | |
|---|---|---|---|---|
| | (for 50 arrays | | Volume | |
| Solutions | by robotics) | | (for 25 arrays by robotics) | |
| Monomer/Apo10 | 66 | μL | 33 | μL |
| Modified streptavidin | 42 | μL | 21 | μL |
| 8% Darocur in DMSO | 12 | μL | 6 | μL |
| Total | 120 | μL | 60 | μL |

2) The solution was mixed by gentle pipetting up and down.
3) The tube was capped and briefly centrifuged on a tabletop centrifuge to remove any bubbles.
4) The solution was allowed to sit at room temperature for 10 minutes before spotting, as this pre-spotting incubation appeared to improve gel performance on the array.

### Spotting Hydrogel Solution on Carbon Arrays and UV Curing

### (i) Array Preparation

Carbon array sheets were removed from their bag, and the array side was blown with pressured air (e.g., CRC Duster) to remove dust and lint. Sheets were carefully cut into individual arrays, and lot numbers were dated on the back of each array.

### (ii) UV Device Preparation

The UV curing device (e.g., Hand-Held UV device, Model UVM-57, Mid Range UV-302 nm lamp, UVP, Upland, CA) was turned on 5-10 minutes before array spotting to warm up the lamp. The UV light was maintained facing down towards the lab bench.

(iii) Storage Solution Preparation 50 mM histidine solution (with 0.05% Proclin) was added to clean large petri dishes for storing spotted arrays after curing.

### (iv) Spotting With BioDOT (or Similar Robotic Spotter)

The BioDOT instrument was prepared, and arrays were placed on the alignment plate. 20 nL of the hydrogel spotting solution was spotted onto each electrode of the carbon array.

### (v) UV Curing

Immediately after spotting, the carbon array was placed on an array guider, and the hand-held UV device (with UV light on) was placed on top of the array guider for 10 seconds to cure the hydrogel.

### (vi) Post-Curing Storage

After 10 seconds of UV exposure, the UV device was removed, and the UV-treated array was placed in a petri dish filled with 50 mM histidine solution, ensuring the electrodes (now with cured hydrogel) were facing down. It is recommended for the array to soak in the 50 mM histidine solution for more than 1 hour before use.

### Conclusions

The present invention, particularly as further developed and disclosed herein, provides significant advancements in the field of rapid and multiplexed nucleic acid detection. The methods and devices described offer substantial improvements in speed, efficiency, breadth of applicability, and predictability by focusing on the specific architecture and preparation of hydrogel microgel deposits utilized within an electrophoretic array system.

A central feature of the invention is the use of hydrogel microgel deposits which function as highly optimized “search stations” or “super-librarians” within an electrophoretic array “library.” These microgel deposits are specifically fabricated to possess a defined three-dimensional porous hydrogel matrix structure. This structure is achieved, for example, by employing a porogen (such as APO 10) during an in-situ polymerization process involving specific monomers (like acrylamide and BIS), an affinity-binding molecule (like modified streptavidin for robust probe/primer anchoring), and a controlled curing step (like UV curing with a photoinitiator such as Darocur 4265), as detailed in the hydrogel preparation protocol (Example 6).

This carefully engineered microgel architecture provides a highly controlled and optimized localized reaction environment. The porogen-derived pore network within the microgel is specifically configured (as evidenced by its ability to transport nucleic acid molecules up to at least 800 base pairs in length) to allow efficient entry of various target molecules (different “books”) and necessary reagents (“search tools” like enzymes and primers) into the “librarian's workspace.” This enhanced accessibility contributes to efficient initial target recognition and complex formation for a variety of targets. Furthermore, the defined structure facilitates enhanced reaction kinetics for subsequent steps like ligation and Rolling Circle Amplification (RCA), and aids in the effective concentration and retention of amplicons (“photocopies”) within the microgel. Such features lead to more robust and predictable outcomes compared to systems employing less defined surfaces or bulk solution reactions.

The detailed hydrogel preparation protocol provided herein (Example 6), combined with the comprehensive descriptions of the assay steps (illustrated in FIGS. 4A-7J) and the application of established principles of RCA and electrokinetics, furnishes a clear and enabling disclosure for achieving these optimized microgels and their functional benefits.

The enhanced capabilities of the present invention are further underscored by the inclusion of new working Examples 3, 4, and 5 (FIGS. 10, 11, and 12). These examples demonstrate the successful rapid detection of additional, structurally different bacterial DNA targets, namely Klebsiella pneumoniae and Listeria monocytogenes, including their simultaneous (multiplex) detection. This expansion beyond the meningitis panel (Examples 1 and 2, FIGS. 8 and 9) showcases the broader applicability of the system, illustrating that the “electric library” with its specialized “search stations” can indeed handle requests for different types of “books” efficiently. This robustly supports the methods and devices for detecting a “plurality of potentially different pre-defined nucleic acid target molecules”. The ability to pre-anchor different target-specific components within distinct, structurally optimized microgels on the same array facilitates true multiplexing, where each “librarian” at its known “desk” (microgel location) effectively searches for its designated “book title” (target sequence).

The claims defining the present invention clearly recite the essential steps, including the utilization of electric fields to drive the target into the porous microgel structure, the formation of a ligation-competent complex, the crucial ligation of the linear RCA probe to form a circular template within the hydrogel microgel deposit, the performance of RCA, and detection within a very rapid timeframe (typically less than 20 minutes, and in certain embodiments, between 8 and 15 minutes). The role of the immobilized affinity-binding molecule (e.g., modified streptavidin) within the hydrogel matrix is also clearly linked to the versatile and robust anchoring of various biotinylated target-specific components (such as capture probes, primers, and linear RCA probes), providing a flexible system for configuring the “search stations.”

In summary, the present invention provides a significant step forward by detailing the fabrication and functional advantages of specifically structured hydrogel microgel deposits. These engineered reaction hubs, with their porogen-derived pore networks and integrated affinity-binding systems, when combined with electrophoretic arrays and RCA, lead to a nucleic acid detection platform that is not only rapid and sensitive but also robustly applicable to the multiplexed detection of diverse targets. The disclosed methods and devices offer a clear and enabled solution for advanced molecular diagnostics. CLAIMS

1. An electrophoretic array device for rapid, multiplex detection of a plurality of different pre-defined potential nucleic acid target molecules, the device comprising:
(1) a substrate comprising a plurality of electrodes defining discrete electrode locations; and
(2) a plurality of hydrogel microgel deposits immobilized at said discrete electrode locations, each said hydrogel microgel deposit defining a microgel region and comprising a three-dimensional hydrogel matrix, said matrix comprising:
(a) a cross-linked polymer;
(b) an immobilized affinity-binding molecule; and
(c) a porogen-derived pore network defining interconnected void spaces within said matrix;
wherein said hydrogel matrix is configured to provide a localized reaction environment for enzymatic reactions and rapid molecular transport of nucleic acid molecules up to at least 800 base pairs in length, and for concentration of reaction products; and
wherein at least a subset of said hydrogel microgel deposits each contain target-specific components pre-anchored within its said hydrogel matrix via said affinity-binding molecule, said target-specific components being different between at least two microgel deposits in said subset, enabling specific capture or initiation of nucleic acid amplification for said plurality of potentially different pre-defined nucleic acid target molecules.

2. The device of claim 1, wherein said plurality of electrodes comprise carbon electrodes.

3. The device of claim 1, wherein said hydrogel microgel deposits are formed by UV curing a spotted polymerisable hydrogel solution that includes monomers for said cross-linked polymer, a porogen for forming said porogen-derived pore network, said affinity-binding molecule or precursors thereof, and a photoinitiator.

4. The device of claim 3, wherein said monomers comprise acrylamide and N,N′-methylenebisacrylamide (BIS), said cross-linked polymer is polyacrylamide, and said affinity-binding molecule is modified streptavidin.

5. The device of claim 1, wherein said hydrogel matrix of at least a portion of said deposits contains pre-anchored target-specific components specific for nucleic acid targets selected from the group consisting of Neisseria meningitidis, Klebsiella pneumoniae, Listeria monocytogenes, Haemophilus influenzae group B, Escherichia coli, Group B Streptococcus, and combinations thereof.

6. The device of claim 1, wherein said hydrogel microgel deposits are in a dehydrated state suitable for room-temperature storage and are configured to rehydrate upon contact with an aqueous solution.

7. A method of rapidly detecting in a solution the presence of at least one nucleic acid target molecule from a plurality of potentially different pre-defined nucleic acid target molecules, the method comprising:
(A) Providing an electrophoretic array device according to claim 1;
(B) Introducing said solution, potentially containing one or more of said plurality of different pre-defined nucleic acid target molecules, to said electrophoretic array device, causing said solution to contact said hydrogel microgel deposits having said porogen-derived pore network defining interconnected void spaces;
(C) Utilizing electric fields to drive said at least one nucleic acid target molecule present in said solution into the interconnected void spaces of the porous hydrogel matrix of one or more specific hydrogel microgel deposits of said device containing corresponding nucleic acid target-specific components;
(D) Within at least one said specific hydrogel microgel deposit, forming a ligation-competent complex involving a said nucleic acid target molecule, a linear rolling circle amplification (RCA) probe specific to said nucleic acid target molecule, and at least one primer, wherein said complex formation occurs within the localized reaction environment provided by said hydrogel microgel deposit;
(E) Ligating said linear RCA probe within said complex to form a circular RCA probe template within said hydrogel microgel deposit;
(F) Performing rolling circle amplification using said circular RCA probe template within said hydrogel microgel deposit by introducing at least a polymerase enzyme and nucleotides, wherein electric fields are applied to said plurality of microgel regions during said rolling circle amplification to enhance reaction kinetics within the porous hydrogel matrix and concentrate generated amplicons within said hydrogel microgel deposit; and
(G) Detecting the presence of amplified RCA products concentrated within said at least one hydrogel microgel deposit, thereby detecting the presence of said at least one nucleic acid target molecule, wherein said detecting is completed within a time period of between approximately 8 minutes and 20 minutes from said introducing said solution.

8. The method of claim 7, wherein the cross-linked polymer of the hydrogel matrix of the device is polyacrylamide formed from acrylamide and N,N′-methylenebisacrylamide (BIS) monomers.

9. The method of claim 7, wherein the immobilized affinity-binding molecule in the hydrogel microgel deposits of the device is modified streptavidin.

10. The method of claim 7, wherein the hydrogel microgel deposits of the device are formed by UV curing a polymerizable solution containing a photoinitiator.

11. The method of claim 7, wherein said porogen-derived pore network defining interconnected void spaces in the hydrogel microgel deposits of the device enhances the accessibility of said target-specific components pre-anchored within said hydrogel matrix to said at least one nucleic acid target molecule.

12. The method of claim 9, wherein the target-specific components are biotinylated oligonucleotides selected from the group consisting of target-specific capture probes, primers specific to a unique barcode sequence present on a corresponding linear RCA probe, and target-specific linear RCA probes, anchored via said modified streptavidin.

13. The method of claim 7, wherein said target-specific components pre-anchored within said hydrogel matrix in at least one microgel region of the device comprise target-specific capture probes.

14. The method of claim 7, wherein said target-specific components pre-anchored within said hydrogel matrix in at least one microgel region of the device comprise primers specific to a unique barcode sequence present on a corresponding linear RCA probe.

15. The method of claim 14, wherein said primers are pre-hybridized to said linear RCA probes within said hydrogel matrix of the device prior to introducing said solution.

16. The method of claim 7, wherein said target-specific components pre-anchored within said hydrogel matrix in at least one microgel region of the device comprise target-specific linear RCA probes.

17. The method of claim 7, wherein said target-specific components pre-anchored within said hydrogel matrix in at least one microgel region of the device comprise forward primers and reverse primers for said RCA.

18. The method of claim 7, wherein said solution introduced to said array device comprises said linear RCA probe and said at least one primer, and wherein said target-specific components pre-anchored within said hydrogel matrix of the device comprise target-specific capture probes configured to bind a complex formed by said nucleic acid target molecule and said linear RCA probe.

19. The method of claim 7, wherein said plurality of potentially different pre-defined nucleic acid target molecules are selected from the group consisting of DNA from Neisseria meningitidis, Klebsiella pneumoniae, Listeria monocytogenes, Haemophilus influenzae group B, Escherichia coli, Group B Streptococcus, and combinations thereof.

20. The method of claim 7, wherein said method allows for simultaneous detection of at least two different pre-defined nucleic acid target molecules from said plurality in different microgel regions of the device.

21. The method of claim 7, wherein the hydrogel microgel deposits of the device are dehydrated after formation and prior to introducing said solution, and rehydrate upon contact with said solution.

22. The method of claim 6, wherein said detecting comprises fluorescence detection.

23. The method of claim 7, wherein said detecting is completed within a time period between 8 minutes and 15 minutes from said introducing said solution.

24. The method of claim 7, wherein the introducing said polymerase enzyme and nucleotides occurs after said ligating step.
